(19) United States
(12) Patent Application Publication
(10) Pub. No.: US 2008/0300590 A1
(43) Pub. Date: Dec. 4, 2008

(54) APPARATUS AND METHODS FOR MULTIPOLAR TISSUE WELDING

(75) Inventors: Ken Horne, San Francisco, CA (US); Venkata Vegesna, Sunnyvale, CA (US); Hanson S. Gifford, Woodside, CA (US)

Correspondence Address: Takahiro Miura;Oblon, Spivak, 1940 Duke Street, Alexandria, VA, 22314, US

(73) Assignee: Cierra, Inc., Redwood City, CA (US)

(21) Appl. No.: 11/951,478

(22) Filed: Dec. 6, 2007

Related U.S. Application Data
(60) Provisional application No. 60/869,049, filed on Dec. 7, 2006.

(51) Int. Cl. A61B 18/12

(52) U.S. Cl. 606/35

(57) ABSTRACT

Apparatus, systems and methods of welding and coagulating tissue utilize a combination of monopolar and bipolar delivery of RF energy. This method is referred to as multipolar RF delivery and includes bringing a treatment apparatus having first and second electrodes to a treatment site. A first potential is applied to the first electrode and a second potential lower than the first is delivered to the second electrode. This results in current flow from the first electrode through the tissue to the second electrode and then through the tissue to a ground electrode. Current also flows from the first electrode through the tissue to the ground electrode and current may also flow from the first electrode through the tissue to the second electrode and return directly to the ground electrode.

### CROSS-REFERENCES TO RELATED APPLICATIONS

The present application claims the benefit of U.S. Provisional Application No. 60/869,049 (Attorney Docket No. 022128-001500US), filed Dec. 7, 2006, the entire contents of which are incorporated herein by reference.

### BACKGROUND OF THE INVENTION

1. Field of the Invention

The present invention generally relates to medical apparatus, systems and methods. More specifically, the invention relates to energy based heating, bonding or welding of soft tissue and, more particularly, to an apparatus, system and methods for controllably delivering energy to tissue for welding thereof.

Radiofrequency (RF) energy has been used for many years in electrosurgical instruments to cut, ablate, coagulate, heat, shrink, desiccate and cauterize various tissues of the body. RF energy ranges in frequency from 3 KHz up to 300 GHz, although many medical applications operate in the range from about 100 KHz to about 5 MHz. RF energy has traditionally been delivered in medical applications using either a monopolar or bipolar modality. In monopolar applications, a voltage source is applied to the treatment site through a single electrode or probe, causing an electrical current to flow through the tissue to a return electrode maintained at ground potential and then back to the power source. Often the return electrode is a plate that the patient lies on during the procedure or the return electrode may be an electrode adhesively attached to the patient's skin. Monopolar delivery of energy tends to focus on the path of tissue between the source and return electrodes and hence monopolar applications are best for affecting heating close to the probe and to some depth therefrom. Some challenges with this method include the fact that skin burns can occur when there is poor contact between the body and the return electrode during energy application.

The bipolar modality on the other hand employs a pair of electrodes. For example, tissue may be grasped between a pair of electrodes, often forceps, and the electrodes are connected to an RF energy source. Current flows between the electrodes and through the tissue grasped therebetween resulting in heating of the tissue. Bipolar delivery of energy tends to heat lateral areas of tissue more effectively than monopolar systems, but has limited depth of heating.

The waveform of the RF energy may also be varied in different RF applications. For example, a continuous single frequency sine wave is often used in cutting applications. This waveform results in rapid heating resulting in tissue cells boiling and bursting which creates a fine line in the tissue, as required for a clean incision. On the other hand, for coagulation, a sine wave is turned on and off in rapid succession, resulting in a slower heating process thereby causing coagulation. The duty cycle (ratio of on time to off time) can therefore be varied to control heating rates. For coagulation of tissue, optimal tissue temperature is about 50-55° C., where denaturation of albumens occurs in the tissue. The denaturation of the albumens results in the “unwinding” of globular molecules of albumen and their subsequent entangling which results in coagulation of the tissues. Once the tissue is treated in this way, the tissue can be cut in the welded area without bleeding. This allows the targeted tissue to be cut without bleeding. This process is commonly referred to as bipolar coagulation.

Tissue welding generally comprises bringing together edges of an incision to be bonded, compressing the tissue with a bipolar tool and heating the tissue by the RF electric current flowing through them. One of the major differences between tissue welding procedures and coagulation with the purpose of hemostasis (limiting bleeding) is that tissue welding requires conditions which allow for the formation of a common albumen space between the tissue to be bonded before the beginning of albumen coagulation. If such conditions are not present, coagulation will take place without a reliable connection being formed.

Problems that can occur during the tissue welding process include thermal damage to adjacent structures, over-heating of tissue and under-coagulation. Over-heating of tissue results in delayed healing, excessive scarring, tissue charring/destruction, and tissue sticking to the electrosurgical tool. If tissue sticks to the electrosurgical tool upon removal, the tissue can be pulled apart at the weld site, adversely affecting hemostasis and causing further injury. Under-coagulation can occur if insufficient energy has been applied to the tissue. Under-coagulation results in weak and unreliable tissue welds, and incomplete hemostasis.

Precise control of the welding process while avoiding excessive thermal damage, over-heating or under-coagulation is a difficult process, particularly when attempting to weld tissue of varying structure, thickness and impedance. It is particularly important to control these variables when welding organs, such as cardiac tissues, since recovery of physiologic function of such organs is a critical requirement. In addition, creating a viable automatic control system to control the variables is particularly important to create a procedure that can be relied upon by a physician to weld the tissue in a way that maintains organ viability following the procedure. For example, vessels or other vascularized tissue parts, such as cardiac tissues, that have been excessively heated typically do not recover and lose functionality. Control of heating can be especially important when heating in a complex organ, or a layered tissue structure, where tissue thickness and the make up of the tissue (collagen content, type of cellular structure, etc.) varies within the targeted region.

Prior attempts to automate the control of tissue coagulation have been taught. For example, temperature measurement devices have been included with or integrated into devices to provide temperature feedback to the energy application device to prevent over-heating the tissue, thereby avoiding excessive heat application that results in unwanted tissue damage. However, in a complex organ, or a layered tissue structure, use of built-in temperature sensors may only provide limited feedback at a localized site around the thermocouple but not allowing for accurate information about the status of the inner layers of the tissue between the electrodes where a weld or connection is desired to be formed.

Several references have suggested various methods of using the tissue impedance and a minimum tissue impedance value to define a point when coagulation is complete and tissue heating should be discontinued. Other references suggest use of a relationship between tissue impedance and current frequency to detect a point of coagulation. These methods, however, do not provide effective tissue bonding solutions for use in surgical procedures and specifically lack the ability to adapt to varying tissue types and thickness during the welding procedure.

It would therefore be desirable to provide an electrosurgical system and method suitable for tissue bonding which allows for adaptation to varying tissue types, structure, thickness, and impedance without over-heating, to provide a reliable tissue connection or weld at the target site. Such a system and method would significantly reduce the time needed for surgical procedures involving tissue welding by eliminating the need for equipment adjustment during the welding process, while increasing the predictability of the outcome. The present invention discloses an improved heating and welding procedure for biological tissue utilizing RF energy which overcomes some of the shortcomings of existing tissue heating and welding systems.

2. Description of Background Art

Prior patents and publications describing various tissue heating, welding and coagulating systems include: U.S. Pat. Nos. 4,532,924; 4,590,934; 5,620,481; 5,693,078; 6,050,994; 6,325,798; 6,893,442; 7,094,215; 2001/0020166; 2002/0156472; 2006/0009762; 2006/0079887; and 2006/0173510.

### BRIEF SUMMARY OF THE INVENTION

The present invention provides apparatus, systems and methods for heating, welding and coagulating biological tissue, including anatomic defects such as a patent foramen ovale as well as atrial and ventricular septal defects, left atrial appendage, patent ductus arteriosis, blood vessel wall defects and the like.

In a first aspect of the present invention, a tissue coagulation system includes a power source, a ground electrode and a plurality of active electrodes connected in parallel to the power source. For purposes of clarity since two types of electrodes are referenced in this specification, active electrodes may be referred to simply as electrodes for the sake of brevity and are distinguished from return electrodes or ground electrodes, both at ground potential. The ground electrode is electrically coupled with the power source through the tissue and is typically remote from the active electrodes. The system also includes at least one resistor or diode connected in series with one of the plurality of active electrodes so that the potential applied to one electrode is higher than the potential applied to another electrode. Thus, the voltage drop across one of the active electrodes may be different from the voltage drop across another of the active electrodes. The resistor or diode may be variable and some embodiments may have a resistor or diode control circuit which controls the variable resistor or diode in order to control the path of the current flow between the two active electrodes.

In another aspect of the present invention, a tissue coagulation and welding system comprises a plurality of active electrodes, a ground electrode generally remote from the active electrodes and a plurality of power sources. Each of the power sources is electrically coupled to an active electrode such that the voltage drop across one of the active electrodes is different from the voltage drop across a different one of the active electrodes. Each power source is also usually electrically coupled to one ground, often through the tissue to the ground electrode.

In another aspect of the present invention, a tissue coagulation system comprises a power source, a ground electrode electrically coupled with the power source through the tissue and a plurality of active electrodes connected in parallel to the power source. The electrical characteristics of adjacent active electrodes are such that the voltage drop across one active electrode is different from the voltage drop across another active electrode. Some systems may also comprise at least one electrode or series of electrodes connected in series with one of the active electrodes. Often total power applied to the tissue is less than 100 Watts and sometimes it is less than 50 Watts.

In yet another embodiment of the present invention, a tissue coagulation system comprises a power source, a ground electrode generally remote from the active electrodes and electrically coupled with the power source through the tissue, a plurality of active electrodes connected in parallel to the power source and a resistor-capacitor circuit controlling a phase of voltage supplied by the power source connected to at least one of the active electrodes such that a different phase voltage is supplied to at least two different active electrodes.

In some embodiments, the resistor-capacitor (RC) circuit includes a plurality of RC circuits with one RC circuit connected to each of the active electrodes such that the phase of voltage supplied to each active electrode is different. A different RC circuit may be connected to adjacent active electrodes such that the phase of voltage supplied to adjacent active electrodes is unique. Some embodiments may have a plurality of power sources and adjacent active electrodes that are connected to different power sources. A control circuit may be used to control operation of the power source or sources and is also used to control operation of the RC circuit. The control circuit may be used to selectively control the RC circuit so as to vary the amount or specific portion of current from traveling from one active electrode to another active electrode. The control circuit may also selectively control the RC circuit so as to vary over time the amount of current traveling from one active electrode to another active electrode. Other circuits control operation of the power source or sources and control operation of the RC circuit so that the control circuit selectively controls the RC circuit so as to vary in response to a detected impedance or temperature, the amount of current from traveling from one active electrode to another active electrode.

In still another embodiment of a tissue coagulation welding system, the system comprises a plurality of active electrodes, a ground electrode generally remote from the active electrodes and a plurality of power sources electrically coupled with the ground electrode through the tissue. The power sources are typically electrically coupled to each active electrode and a frequency of voltage supplied by at least two of the power sources are different such that the voltage drop across one active electrode is different from the voltage drop across a different active electrode.

Often an amount of current flow from the power source travels from one of the active electrodes through the tissue to another active electrode and then either through the tissue to the ground electrode or directly back to the ground electrode. Current also may flow from the power source to one of the active electrodes and then directly from the active electrodes through the tissue to the ground electrode.

In some embodiments, the system further comprises an impedance measuring circuit operably connected to the power source or power sources that measures the impedance of the tissue. Systems may also comprise a catheter having an elongated tubular housing that is sized to fit within the venous system of a mammal. In this embodiment, the active electrodes are typically housed within the elongate tubular housing in an undeployed state. Other embodiments may include a circuit controlling operation of the power source or power sources or a control circuit operably coupled to the impedance measuring circuit that controls operation of the power source. The control circuit discontinues the flow of power to the active electrodes when the impedance measured by the impedance measuring circuit exceeds a threshold value. The impedance control circuit may set the threshold value to equal an initially measured value, initiating flow of power to the active electrodes, and the flow of power to the active electrodes is discontinued when impedance measured by the measuring circuit exceeds the threshold value.

The control circuit may iterate through at least two power cycles where the control circuit sets the threshold value as an impedance value measured at the beginning of each power cycle. The control circuit also may initiate a flow of power to the active electrodes and then discontinue power for a predetermined rest period when an impedance value measured by the impedance measuring circuit exceeds the threshold impedance value stored at the beginning of that power cycle. The control circuit may discontinue power and terminate iteration through any further power cycles once power has been applied for a predefined duration regardless of an impedance value measured by the impedance measuring circuit.

Often, in the tissue coagulation system, the plurality of active electrodes comprises N-number of active electrodes and the at least one resistor or diode comprises N-number of variable resistors or diodes, with one of the variable resistors or diodes connected in series with each of the N-number of active electrodes. The control circuit controls resistance of the variable resistors or the voltage drop across the diode so as to control the relative flow of current between the active electrodes. The control circuit may include a resistor or diode control circuit that controls the plurality of variable resistors or diodes to control the path of current flow between the active electrodes. The control circuit also can discontinue the flow of power to the active electrodes when impedance measured by the impedance measuring circuit exceeds a threshold value. Often, the impedance measuring circuit measures an initial impedance of the tissue and the control circuit discontinues the flow of power to said active electrodes when measured impedance exceeds the initial impedance. The control circuit may iterate through at least two power cycles and the control circuit stores an impedance value measured at the beginning of each power cycle, then applies power to the active electrodes and discontinues power to the active electrodes for a predetermined rest period when measured impedance exceeds the impedance value stored at the beginning of the power cycle. The control circuit may discontinue power and terminate iteration through any further power cycles once power has been applied for a predefined duration regardless of an impedance value measured by the impedance measuring circuit.

In some embodiments of the system, the control circuit selectively controls the power sources so as to vary the amount of current traveling from one active electrode to another active electrode. The control circuit may also selectively control the power sources so as to vary over time the amount or specific portion of current from traveling from one active electrode to another active electrode. The coagulation system may further comprise a circuit for controlling operation of the power sources, the circuit selectively controlling the power sources to vary in response to a detected impedance with an amount or specific portion of current from traveling from one active electrode to another active electrode.

In another aspect of the present invention, an apparatus for coagulating tissue comprises an elongate flexible member having a proximal end and a distal end. A plurality of electrodes are disposed near the distal end of the elongate flexible member and they are adapted to being coupled in parallel to a power source, the plurality of electrodes are also adapted so that a resistor or diode connected in series with one of the electrodes results in a voltage drop across one of the electrodes different from a second voltage drop across another electrode.

In another aspect of the present invention, an apparatus for coagulating tissue comprises an elongate flexible member having both proximal and distal ends and a plurality of electrodes disposed near the distal end of the elongate flexible member. The electrodes are coupleable in parallel to a power source and are adapted to also be coupled to a RC circuit controlling a phase of voltage supplied to at least one of the electrodes such that a different phase voltage can be supplied to at least two different electrodes. In still another aspect of the present invention, an apparatus for coagulating tissue comprises an elongate flexible member having both proximal and distal ends and a plurality of electrodes disposed near the distal end of the elongate flexible member. The electrodes are adapted to be coupled with two or more power sources such that a frequency of voltage supplied by the two or more power sources are different and the voltage drop across one of the electrodes is different from the voltage drop across a different electrode.

Often, the electrodes are active electrodes and the active electrodes are mounted to a resilient housing and a thermocouple may be mounted to the resilient housing and/or a thermocouple may also be mounted on one of the active electrodes. Adjacent electrodes are generally electrically insulated from one another so that current traveling between electrodes passes through tissue. The electrodes may be in any orientation, but can be generally planar and in some cases the surface area of one active electrode is larger than the surface area of another electrode. In some embodiments, the plurality of active electrodes comprise two active electrodes with one active electrode having a surface area at least three times as large as the surface area of the other active electrode. Still, in other embodiments, the plurality of active electrodes comprise two active electrodes with one of the active electrodes comprising two segments which are adjacent to or disposed on either side of the other active electrode. Sometimes, the first active electrode is generally circular in shape and the two segments are arcuate.

In yet another aspect of the present invention, a method for coagulating tissue comprises bringing a treatment apparatus to a tissue treatment site. The treatment apparatus has both proximal and distal ends and first and second electrodes near the distal end. Positioning the first and second electrodes into apposition with tissues of the tissue treatment site allows the treatment apparatus to effectively coagulate the tissue when a potential is applied. Applying a first potential to the first electrode and a second potential lower than the first potential to the second electrode allows current to flow from the first electrode through the tissue to the second electrode and then through the tissue to a ground electrode. Current also flows from the first electrode through the tissue to the ground electrode. Often, current also flows from the first electrode through the tissue to the second electrode and current then returns to the ground electrode.

Sometimes the method further comprises measuring impedance of the tissue and the potential applied to the first and second electrodes may be controlled based on the measured tissue impedance. Other times, the method comprises measuring temperature of the tissue with a thermocouple disposed on either the first or second electrodes or both electrodes and the potential applied to the first and second electrodes is controlled based on the measured tissue temperature. Tissue temperature may be an average value of the temperature measured by two or more thermocouples. In some embodiments, the method further comprises deploying the first and second electrodes from a catheter.

Applying the second potential may include providing a resistor or diode in series with the second electrode so that the second potential is lower than the first potential. Alternatively, applying the first and second potentials may include providing two power supplies. Or, applying the second potential may comprise providing a RC circuit in series with the second electrode so that the second potential is out of phase with the first potential. In still another variation, applying the second potential may include providing the second potential at a frequency different than the frequency of the first potential.

These and other embodiments are described in further details in the following description related to the appended drawing figures.

### BRIEF DESCRIPTION OF THE DRAWINGS

FIG. 1 shows a conventional monopolar electrosurgical system;

FIG. 2 illustrates a conventional bipolar electrosurgical system;

FIG. 3A-3C show schematic diagrams of multipolar electrosurgical systems having varying number of electrodes according to the present invention;

FIG. 4 shows a patent foramen ovale;

FIG. 5 shows a top view of a multipolar electrode according to the present invention;

FIG. 6A illustrates the multipolar electrode of FIG. 5 coupled to a resilient housing;

FIGS. 6B-6D show top, side and front views of the resilient housing depicted in FIG. 6A;

FIG. 7 illustrates a tissue heating and welding system comprising a multipolar electrode coupled to a housing on the distal end of a catheter shaft;

FIGS. 8A-8C show an exemplary embodiment of closing a patent foramen ovale using a multipolar electrosurgical system;

FIG. 9 illustrates an alternative embodiment of a multipolar electrosurgical catheter;

FIGS. 10A-10C illustrate the use of a resistor to apply different potentials across the multipolar electrodes in systems with varying number of electrodes;

FIG. 11 shows multiple power supplies in a multipolar electrosurgical system;

FIGS. 12A-12C show embodiments of the present invention utilizing inherent electrode resistance in multipolar electrosurgical systems having various numbers of electrodes;

FIGS. 13A-13D illustrate the use of phase control in several embodiments of a multipolar electrosurgical system having various numbers of electrodes;

FIGS. 14A-14C illustrate the use of frequency control in several embodiments of a multipolar electrosurgical system having various numbers of electrodes;

FIG. 15 illustrates temperature, power and tissue impedance during tissue welding using a multipolar electrosurgical system; and

FIGS. 16A-16F illustrate the use of various diode circuits in several embodiments of a multipolar electrosurgical system having various numbers of electrodes.

### DETAILED DESCRIPTION OF THE INVENTION

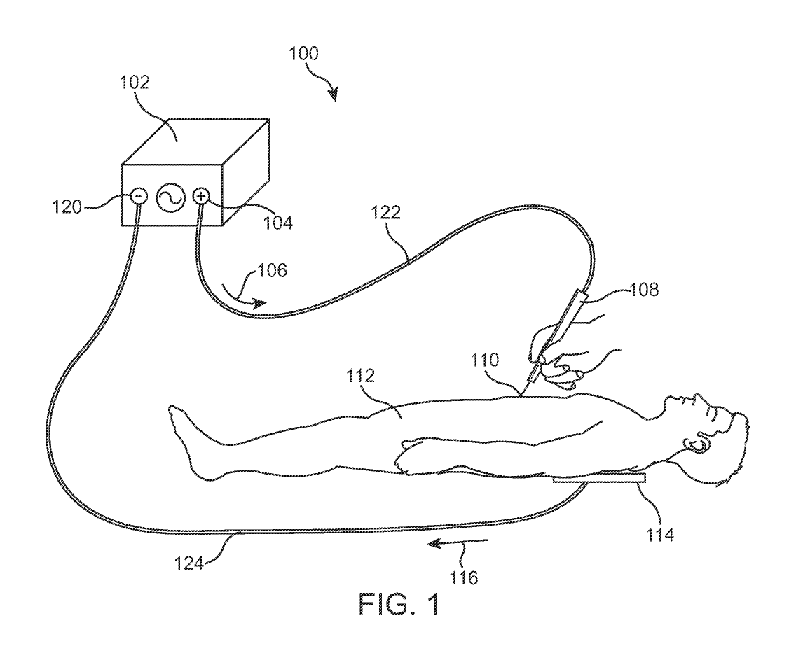

Referring now to FIG. 1, a conventional monopolar electrosurgical system 100 is illustrated. Such systems are often used for heating tissue, cutting, coagulating, desiccating, ablating and welding tissue. In FIG. 1, monopolar electrosurgical system 100 includes a power supply 102, usually a RF power supply and an electrode 108. Electrodes in this specification are active electrodes as distinguished from return electrodes or ground electrodes at ground potential, but may be referred to simply as electrodes for the sake of brevity. A lead 122 couples electrode 108 with the higher potential (positive) terminal 104 of RF power supply 102. Electrode 108 is manipulated by a physician during an electrosurgical procedure and the distal tip 110 of electrode 108 directs RF energy to target tissue treatment locations in a patient 112. Electrosurgical system 100 is activated, typically with a footswitch or a switch on electrode 108, and on a positive half cycle of RF power from power supply 102, current flows from RF power supply 102 to electrode 108 along lead 122 in the direction indicated by arrow 106. Current then flows from electrode 108 through the patient 112 toward a return electrode 114 typically located under patient 112. From the return electrode 114, current then flows along lead 124 back to the lower potential, negative terminal 120 of RF power supply 102 in the direction of arrow 116, thereby completing the circuit. On a negative half cycle of RF power from power supply 102, current flows in the opposite direction.

Monopolar electrosurgical systems such as system 100 illustrated in FIG. 1 are ideal for localized heating around the electrode tip 110. Heating can be provided in a relatively deep but narrow band of tissue. In order to create a wider band of heating, larger electrodes must be used. However, as the size of the electrode increases, the distribution of heat becomes less uniform. A phenomenon known as shielding results in a heating band biased toward the outer perimeter of the electrode and the center section often remains cooler than the edges. Thus, depending on the size of the treatment area, a monopolar RF system may not always achieve satisfactory tissue heating and welding results.

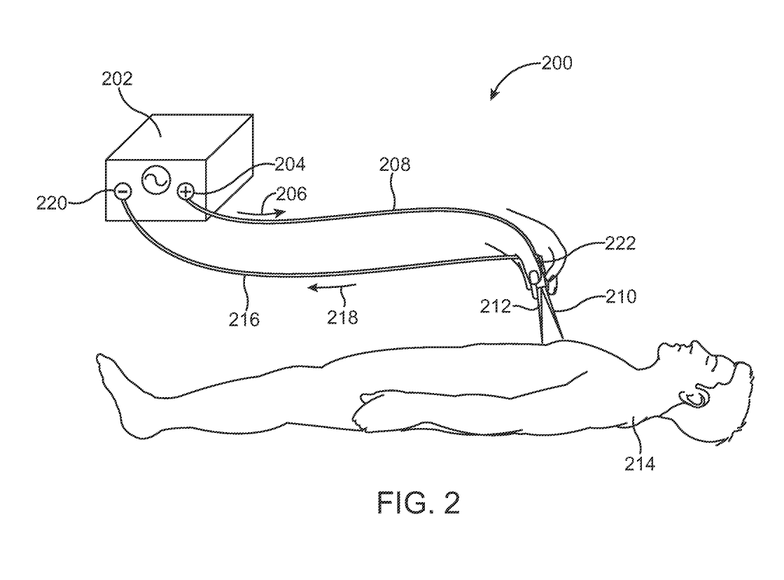

FIG. 2 illustrates a conventional bipolar electrosurgical system 200. Such systems may be used for heating, cutting, coagulating, desiccating, ablating and welding tissue. System 200 includes a RF power supply 202, a pair of leads 208, 216 and a pair of forceps 222 having two electrodes 210 and 212. One arm of forceps 222 forms one electrode 210 which is coupled via lead 208 to one terminal 204 of RF power supply 202. The opposing arm of forceps 222 forms a second electrode 212 and is coupled to the second terminal 220 of RF power supply 202 by lead 216.

In operation, tissue is grasped between electrodes 210, 212 and when activated, during a positive half cycle, current flows from terminal 204 of RF power supply 202 through lead 208 into electrode 210 in the direction indicated by arrow 206. Current then flows from electrode 210 through tissue of the patient 214 which is grasped therebetween to the second electrode 212. Current then flows back to the second terminal 220 via lead 216 in the direction indicated by arrow 218, thus completing the circuit. Current flows in the opposite direction during the negative half of the power cycle.

Bipolar electrosurgical systems such as system 200 in FIG. 2 produce a wider band of heating as compared to monopolar systems. However, only tissue grasped between electrodes is heated and thus the depth of heating into the body is limited.

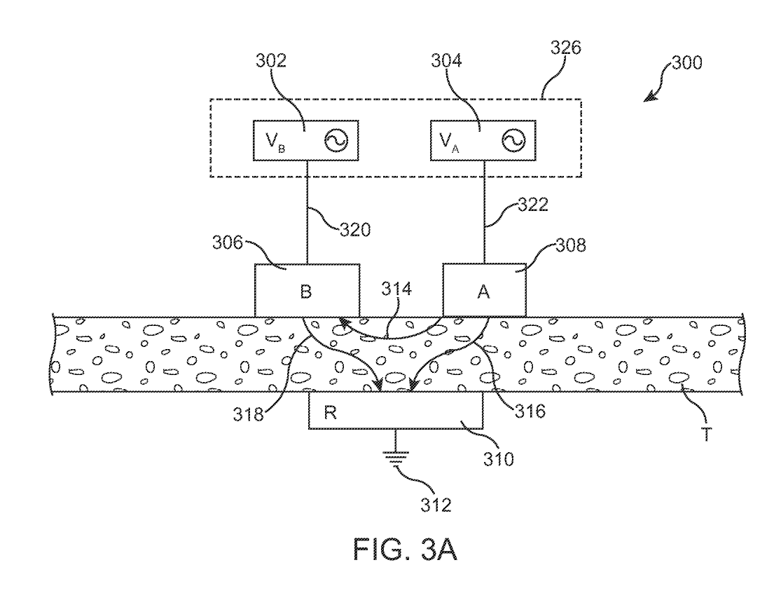

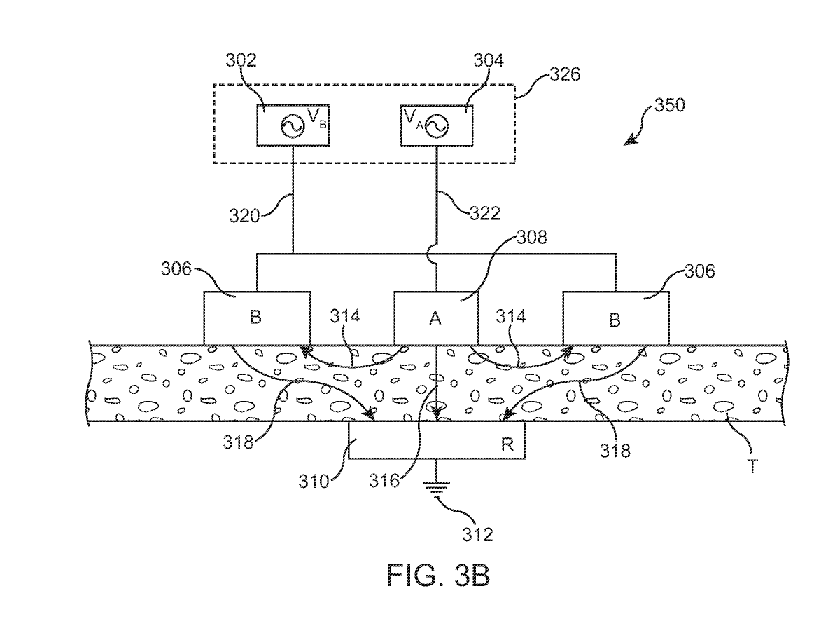

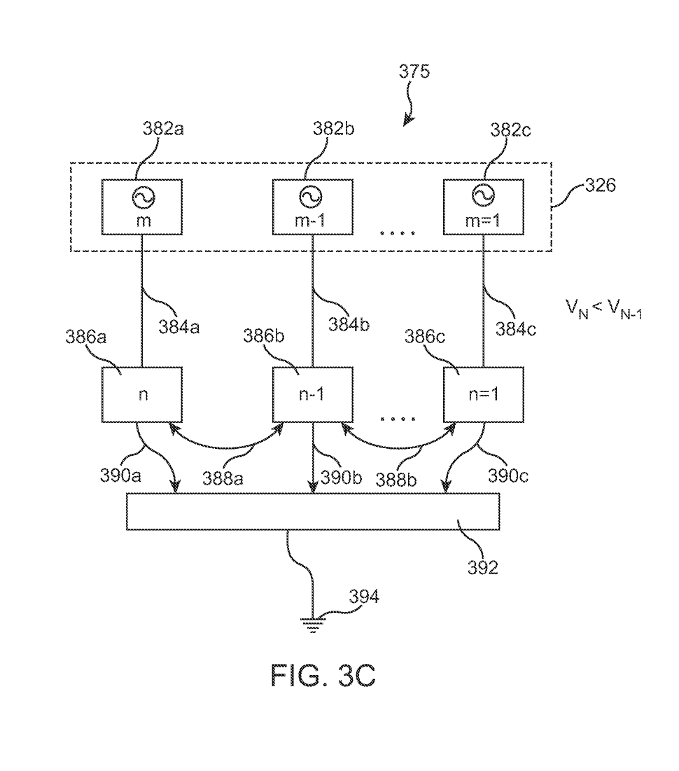

FIG. 3A is a schematic diagram of an improved tissue welding system 300 according to the present invention. The system 300 combines the advantages of both monopolar and bipolar electrosurgical systems to achieve synergistic results. The system 300 of the present invention is able to heat or weld tissue with a wider band of heating having greater depth. Such a system results in better control of the heat applied to tissue thereby producing a better clinical outcome of electrosurgical procedures as well as reducing procedure time because more uniform heating results, requiring few applications of energy. The system 300 includes two or more monopolar electrodes configured such that at least one of the monopolar electrodes operates in a bipolar or quasi-bipolar mode. The system of the present invention is therefore referred to hereinafter as multipolar and will be discussed more fully below. It is important to appreciate that the multipolar system does not utilize true bipolar electrodes.

The multipolar system 300 includes an “A” electrode 308 and a “B” electrode 306 which are both placed in contact with tissue T and a return electrode 310 coupled to ground 312 is also placed in contact with the tissue T remote from electrodes 306, 308. Radiofrequency energy is supplied from a first power supply 304 to electrode 308 via a conductive path 322 and RF energy is supplied from a second power supply 302 to electrode 306 over conductive path 320. The voltage, V_{A} of the first power supply 304 is set to a higher potential than the voltage, V_{B} of the second power supply 302, i.e. V_{A}>V_{B}. The frequency of the RF energy is generally between about 100 KHz and 2 MHz, more preferably between about 100 KHz and about 1 MHz and often between about 300 KHz and about 600 KHz.

FIG. 3A depicts the first and second power supplies 304 and 306 as discrete components; however, the power supplies may both be incorporated into a single power supply 326 as shown in dashed lines in FIG. 3A Notably, power supply 304 may be a first channel from power supply 326 and power supply 306 may be a second channel from power supply 326.

Because the potential of electrode 308 is higher than the return electrode 310 which is at ground potential 312, during the positive half of the power cycle, current will flow along the path of least resistance from electrode 308 through tissue T to return electrode 310 along the path indicated by arrow 316. The tissue near electrode 308 will therefore be heated in a similar manner as a monopolar system. Likewise, current will also flow from electrode 306 through tissue T to return electrode 310 along path 318. Additionally, because the potential applied to electrode 308 is higher than the potential applied to electrode 306, there is a voltage drop across electrodes 306, 308, and current will also flow from electrode 308, through tissue T to electrodes 306 thereby providing a quasi-bipolar effect, although bipolar flow may also result. The flow of current between electrode 308 and electrode 306 is termed quasi-bipolar because in a true bipolar configuration the current would flow from electrode 306 back to the first power supply 304. In contrast, in system 300 current flowing from electrode 308 to electrode 306 then flows through the tissue T to return electrode 310 along the path indicated by arrow 318. During the negative half of the cycle, current will flow in the opposite direction.

The system 300 provides depth of heating from the monopolar flow of current from electrodes 306 and 308 to the return electrode 310. Moreover, a wide band of heating is simultaneously obtained from the quasi-bipolar flow of current between electrodes 306 and 308. The term multipolar is therefore used to describe the simultaneous delivery of both monopolar and quasi-bipolar energy.

Optionally, the potential to the “B” electrode 306 may be multiplexed as required. In this mode, the quasi-bipolar current flow may be switched on and off.

FIG. 3B depicts system 350 which is similar to system 300 but which replaces the single electrode 306 with a pair of adjacent “B” electrodes 306. The multipolar system 350 includes a first “A” electrode 308 and a pair of adjacent “B” electrodes 306 that are electrically coupled to one another and that are on either side of electrode 308. All three electrodes, 306, 308 are placed in contact with tissue, T and a return electrode 310 coupled to ground 312 is also placed in contact with the tissue T remote from electrodes 306, 308. Radiofrequency energy is supplied from a first power supply 304 to electrode 308 via a conductive path 322 and RF energy is supplied from a second power supply 302 to the pair of electrodes 306 over conductive path 320. As previously mentioned, RF power supplies 302, 304 may be discrete or they may be incorporated into a single power supply as indicated by dashed line 326. The voltage, V_{A} of the first power supply 304 is set to a higher potential than the voltage, V_{B} of the second power supply 302, i.e. V_{A}>V_{B}. The frequency of the RF energy is generally between about 100 KHz and 2 MHz, more preferably between about 100 KHz and about 1 MHz and often between about 300 KHz and about 600 KHz.

Because the potential of electrode 308 is higher than the return electrode 310 which is at ground potential, during the positive half of the power cycle, current will flow along the path of least resistance from electrode 308 through tissue T to return electrode 310 along the path indicated by arrow 316. The tissue near electrode 308 will therefore be heated in a similar manner as a monopolar system. Likewise, current will also flow from both electrodes 306 through tissue T to return electrode 310 along path 318. Additionally, because the potential applied to electrode 308 is higher than the potential applied to electrodes 306, there is a voltage drop across electrodes 306 and 308 and therefore current will also flow along path 413 from electrode 308, through tissue T to electrodes 306 thereby providing a quasi-bipolar effect. Current will flow in the opposite direction during the negative half of the power cycle.

As shown in FIG. 3C, a tissue welding system 375 according to the present invention may include n-number of electrodes 386a, 386b, 386c and m-number of power supplies such as RF power supplies 382a, 382b, 382c. The m-number of power supplies 382a, 382b, 382c may be discrete or they may be incorporated into a single power supply as shown by dashed line 326. Electrodes 386a, 386b, 386c are coupled with power supplies 382a, 382b, 382c by conductors 384a, 384b, 384c. Electrodes 386a, 386b, 386c are configured such that the potential at N electrode 386a is less than the potential at N−1 electrode 386b, resulting in a voltage drop across electrodes 386a, 386b, 386c such that current flows between N electrode 386a and N−1 electrode 386b along path 388a, current flows between N−1 electrode 386b and a return electrode 392 coupled to ground 394 along pathway 390b, and current also flows between N electrode 386a and the return electrode 392 to ground 394 along path 390a. Similar potential differences and current flows exist between N−1 electrode 386b and N=1 electrode 386c. Moreover, it should be appreciated that two or more of the n-number of electrodes may be connected to a given one of the m-number of supplies.

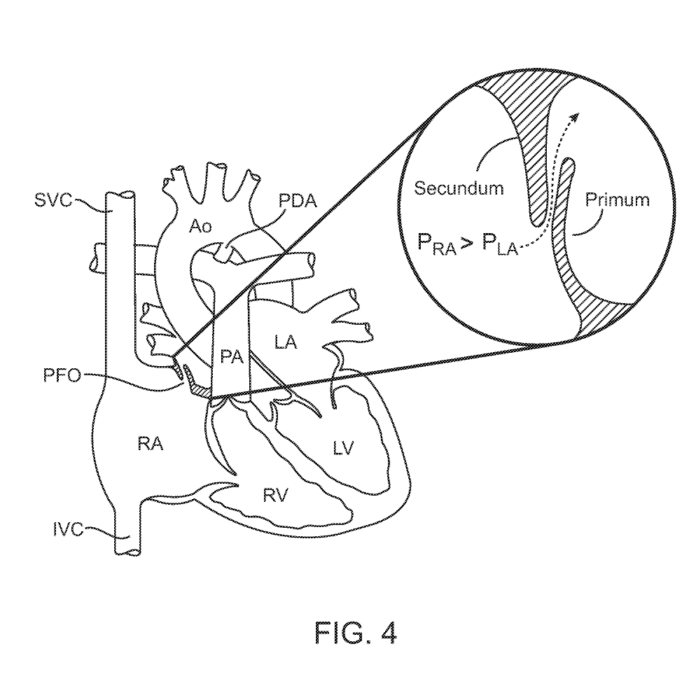

Multipolar RF energy delivery may be applied in specific tissue welding applications. For example, in an exemplary embodiment, tissue welding may be employed to close tissue defects such as a patent foramen ovale (PFO). While this embodiment will be described in the context of closing a PFO, it should be understood that the invention may be employed in any variety of tissue defects such as ventricular septal defects, atrial septal defects, left atrial appendage, patent ductus arteriosis, blood vessel wall defects and other defects having layered and apposed tissue structures as well as generalized tissue heating and welding applications. In those defects where tissue does not overlap, an ancillary tool may be used to approximate the defect prior to application of energy to assist in welding the tissue together. FIG. 4 illustrates a PFO which is a tissue defect caused by the failure of tissues to fuse together during human development, resulting in a patent channel between the right side of the heart and the left side of the heart. PFOs are well documented in the medical and patent literature, such as in U.S. patent application Ser. No. 11/402,489 filed Apr. 11, 2006 (Attorney Docket No. 022128-000730US), the entire contents of which are incorporated herein by reference.

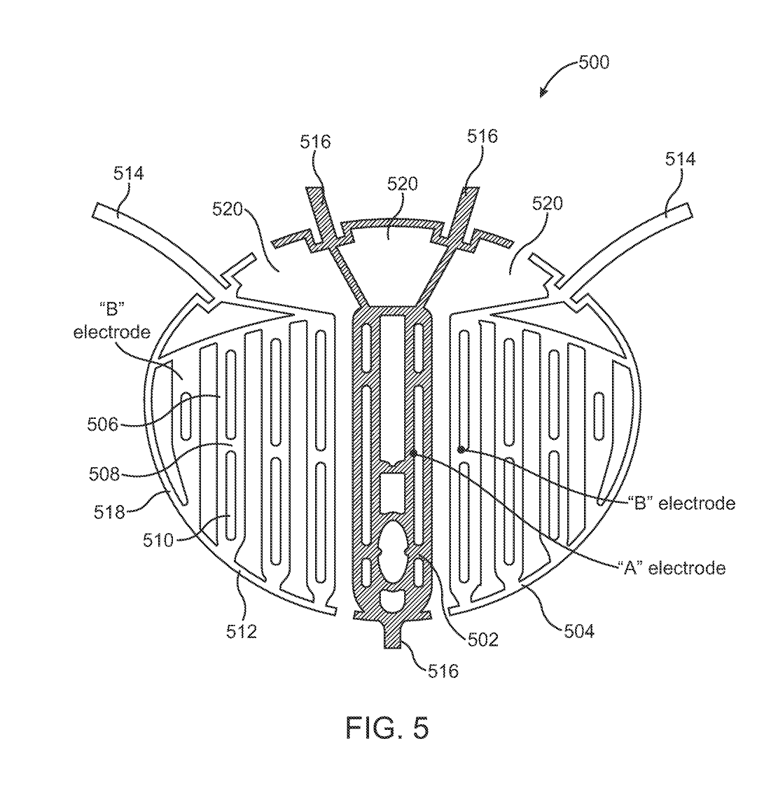

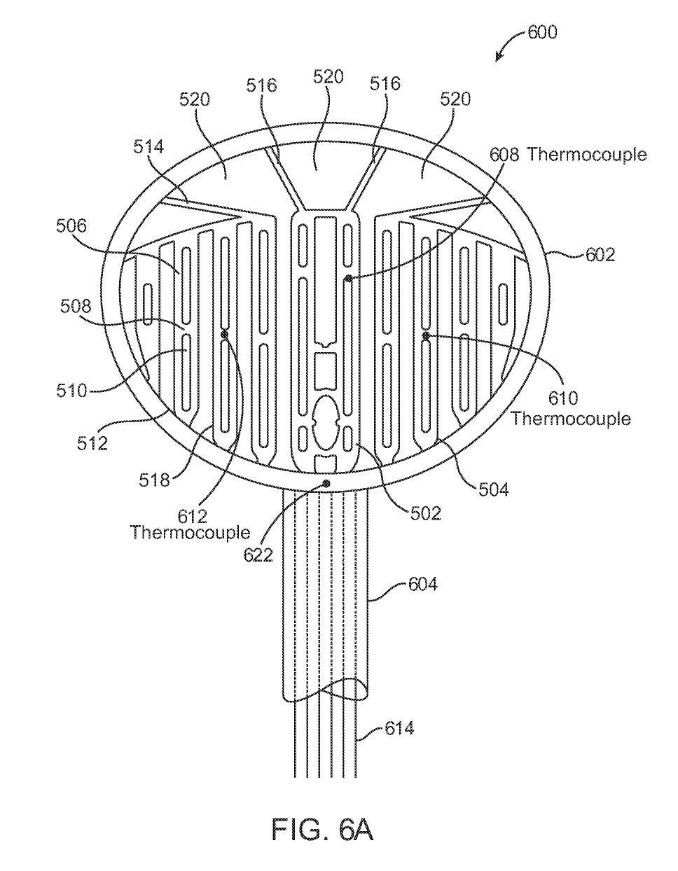

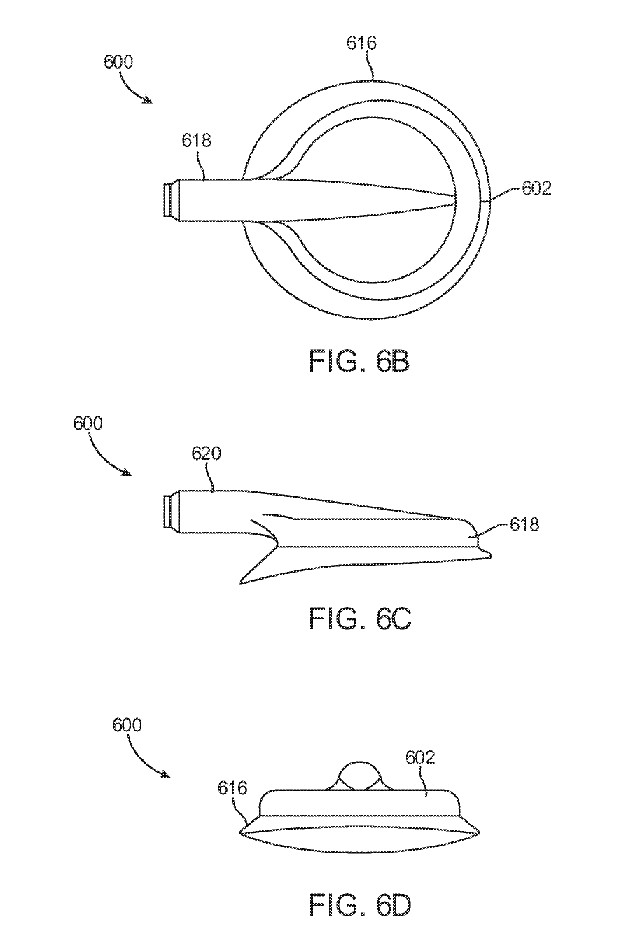

FIG. 5 shows an embodiment of a multipolar electrode that may be used for welding tissue including the tissue layers of PFO thereby closing the defect. In FIG. 5, multipolar electrode 500 comprises three electrodes 502, 504 and 518 forming an overall ovoid shaped pattern. However, one of ordinary skill in the art will appreciate that the multipolar electrode could include as few as two electrodes or could be expanded to include any number of electrodes, depending on the target tissue to be treated. Moreover, one of ordinary skill in the art will appreciate that the invention is not limited to any specific electrode geometry. In the embodiment illustrated in FIG. 5, electrodes 504 and 518 are electrically coupled together while electrode 502 is insulated from the other two electrodes 504, 518. Each electrode 502, 504 and 518 is composed of a series of longitudinal bars 506 with small rectangular gaps 510 between adjacent bars 506. Transverse connectors 508 connect the longitudinal bars 506 together and help provide support to the electrodes 502, 504 and 518. An arcuate perimeter member 512 also couples the longitudinal bars 506 together to further provide support and to electrically couple the longitudinal bars 506 with each other. Support members 514 and 516 extend from electrodes 502, 504 and 518 and allow the electrodes 502, 504, 518 to be coupled with a resilient housing such as in FIG. 6A and also provide a convenient location for attaching conductor wires to the electrodes 502, 504, 518 so that a potential may be applied thereto. Various other gaps 520 are placed between electrodes 502, 504 and 518 in order to allow fluids and/or vacuum to pass through the structure, as will be explained below. The multipolar electrode 500 is typically formed from flat stock such as spring temper stainless steel or superelastic nickel titanium alloys like NiTi so that the multipolar electrode 500 is flexible and may be curled up or folded to reduce its profile prior to use and during delivery. Often, the flat stock is photochemically etched or it may be laser cut, EDM machined or other methods known may be employed to cut the electrode pattern into the flat stock. In addition, such electrode formation may be formed of wire that is bent or heat set to the desired configuration.

FIGS. 6A-6D show the multipolar electrode 500 of FIG. 5 coupled to a resilient housing. FIG. 6A illustrates a bottom view of a multipolar electrode resilient housing 600. The multipolar electrode resilient housing 600 comprises a resilient housing 602 to which electrodes 502, 504 and 518 have been coupled by support members 514, 516 and perimeter member 512. The resilient housing helps provide support for the electrodes 502, 504 and 518. Additionally, the resilient housing 602 is attached to the distal end of a catheter shaft 604. The catheter shaft 604 is used to help deliver the multipolar electrode resilient housing 600 through the vasculature to a target site for tissue heating and welding. In this embodiment, optional thermocouples 608, 610 and 612 are attached to each of the three electrodes 502, 504, 518 in order to help monitor temperature and control the amount of RF energy delivered during treatment. Additionally, an optional thermocouple 622 may be attached to the resilient housing for temperature monitoring and control of energy delivery. Conductor wires 614 run axially in a lumen of catheter shaft 604 from the electrodes 502, 504 and 518 and thermocouples 608, 610, 612 to the proximal end of catheter shaft 604 where they may be connected to a power supply and controller. Additional lumens may be provided in catheter shaft 604 for a guidewire, fluid delivery and for application of vacuum to the treatment tissue in order to assist in positioning of the resilient housing over the targeted tissue and help the resilient housing 602 appose the tissue.

FIG. 6B shows a top view of the multipolar electrode resilient housing 600. In this embodiment, the resilient housing 602 has a soft, compliant flange or skirt 616 that helps resilient housing 602 to seal against tissue during treatment when a vacuum is applied, thereby facilitating apposition of the resilient housing 602 and multipolar electrode 500 against the target treatment tissue. An elongate member 618 represents the transition from resilient housing 602 to a catheter shaft 604. Additionally, the resilient housing 602 has a slightly tapered profile when observed from the side, as in FIG. 6C. The distal tip 618 of resilient housing 602 is the lowest point of the taper and the proximal end 620 of the resilient housing 602 is slightly higher. A front view of resilient housing 602 is seen in FIG. 6D and this view shows the flange or skirt 616 coupled to the resilient housing 602.

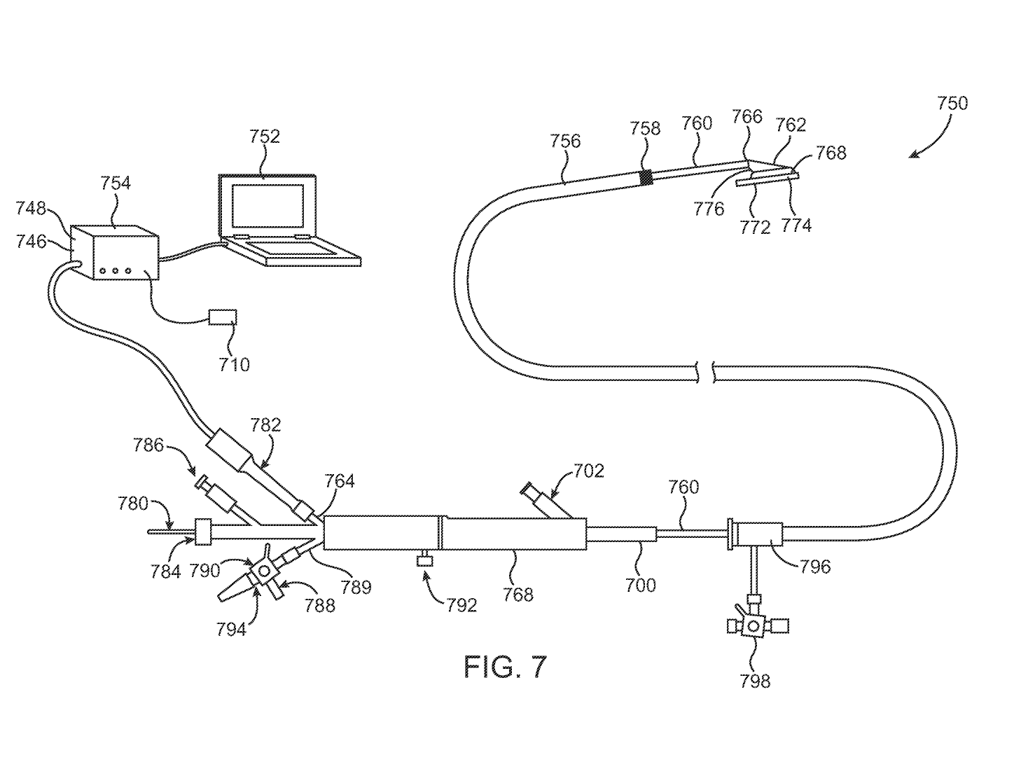

Referring now to FIG. 7, an exemplary system for tissue heating and welding is illustrated. The system of FIG. 7 includes the multipolar electrode 500 of FIG. 5 and the multipolar electrode resilient housing 600 of FIGS. 6A-6D. The system also includes an elongate catheter shaft 760 having a proximal end 764 and a distal end 766, a sheath 756 (or “sleeve”) optionally disposed over at least part of shaft 760, a handle 768 coupled with a proximal end of sheath 756, and a resilient housing 762 coupled with catheter shaft distal end 766. A distal opening 772 for opposing tissue, a multipolar electrode 774 (or other suitable energy transmission member in alternative embodiments for transmitting RF energy to tissues), attachment members 776 (or “struts”) for coupling electrode 774 with resilient housing 762 and for providing support to resilient housing 762, and radiopaque markers (not shown) for coupling attachment members 776 with resilient housing 762 and/or catheter body distal end 766 and for facilitating visualization of device 750. A guidewire 780 is passed through catheter 750 from the proximal end through the distal end. In the embodiment shown, catheter body proximal end 764 includes an electrical coupling arm 782, a guidewire port 784 in communication with a guidewire lumen (not shown), a fluid infusion arm 786 in fluid communication with the guidewire lumen, a suction arm 789 including a suction port 794, a fluid drip port 788, and a valve switch 790 for turning suction on and off.

Fluid drip port 788 allows fluid to be passed into a suction lumen to clear the lumen, while the suction is turned off. A flush port with stopcock valve 798 is coupled with sheath 756. Flush port and stopcock valve 798 allow fluid to be introduced between sheath 756 and catheter body 760, to flush that area. Additionally, sheath 756 has a hemostasis valve 796 for preventing backflow of blood or other fluids. The distal tip of the sheath also has a soft tip 758 for facilitating entry and release of the catheter resilient housing 762 during delivery. The catheter device 750 also includes a collapsing introducer 700 partially disposed in handle 768.

The collapsing introducer facilitates expansion and compression of the catheter resilient housing 762 into the introducer sheath 756. By temporarily introducing the collapsing introducer sheath 700 into introducer sheath 756 the catheter resilient housing 762 may be inserted into introducer sheath 756 and then removed, thereby allowing the introducer sheath 756 to accommodate a larger resilient housing 762 without having to simultaneously accommodate the collapsing introducer 700 as well. The collapsing introducer 700 also has a side port 702 for fluid flushing and a valve (not shown) prevents fluid backflow. Further details on collapsing introducer 700 are disclosed in U.S. patent application Ser. No. 11/403,038 (Attorney Docket No. 022128-000710US), the entire contents of which are incorporated herein by reference. Locking screw 792 disposed in the handle 768 may be tightened to control the amount of catheter shaft 760 movement. A RF power supply 754 is connected to the catheter via the electrical coupling arm 782 and a controller 752 such as a computer is used to monitor and/or control energy delivery. A return electrode or ground pad 710 is also coupled with the power supply 754. In operation, it may also be possible to de-couple the handle from the device if desired, or to remove the handle altogether.

Power supply 754 may also include a circuit 746 controlling operation of the power source 754 and an impedance measuring circuit 748 operably connected to power source 754 capable of measuring tissue impedance. The control circuit 746 may control operation of power source 754, wherein the control circuit 746 discontinues the flow of power to electrodes 774 when impedance measured by circuit 748 exceeds a threshold value. The impedance measuring circuit 748 may set the threshold value to an initially measured value and then initiate power flow to the electrodes 774 until impedance measured by circuit 748 exceeds the set threshold value and power flow is discontinued. In some embodiments, the control circuit 746 iterates through at least two power cycles where the control circuit 746 sets the threshold value to an impedance value measured at the beginning of each power cycle. Power flows to the electrodes 774 and is then discontinued for a predetermined rest period when an impedance value measured by the impedance circuit 748 exceeds the threshold value stored at the beginning of the power cycle. In still other embodiments, the power control circuit 746 may discontinue power and stop iteration through any further power cycles once power has been applied for a predefined duration regardless of an impedance value measured by impedance circuit 748.

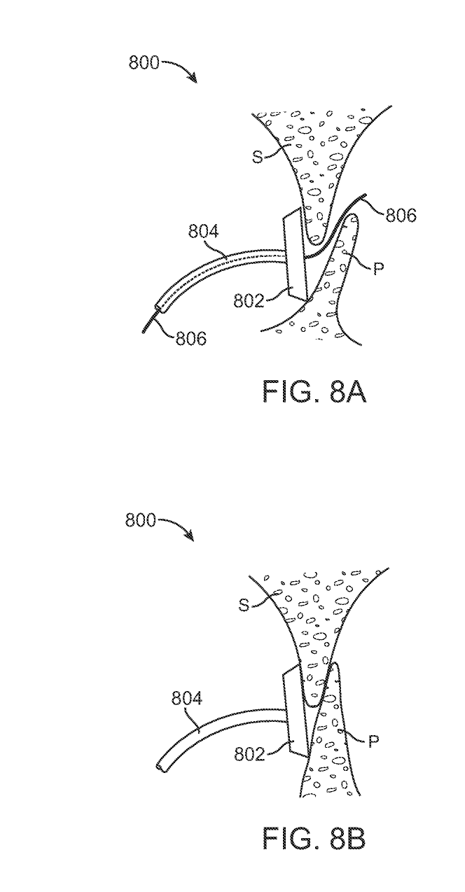

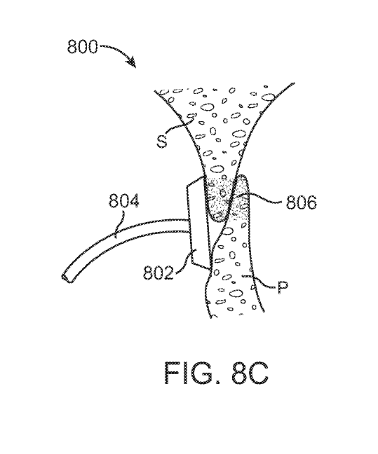

FIGS. 8A-8C illustrate the use of a multipolar electrosurgical catheter in the treatment of a patent foramen ovale. In FIG. 8A, a multipolar electrosurgical catheter 800 having a multipolar electrode 500 (FIG. 5) and a resilient housing 802 similar to multipolar electrode resilient housing 602 in FIGS. 6A-6D are coupled to catheter shaft 804. The electrosurgical catheter 804 is placed into a patient's vasculature by standard introduction techniques such as the Seldinger technique and then advanced through the vasculature into the right side of the heart, adjacent to the septum primum P and septum secundum S tissues of a PFO. In FIG. 8B, a vacuum is applied from the catheter 804 so that resilient housing 802 is apposed with the PFO tissues P, S and the guide wire 806 may be removed so that the primum P and septum S tissue are also apposed against one another. In FIG. 8C, RF energy is delivered to the multipolar electrode in resilient housing 802 using the multipolar energy delivery modality previously discussed, resulting in heating and welding of tissue layers P and S together, thereby closing the PFO tissue defect. The catheter 804 is then removed along with the guide wire 806 from the patient.

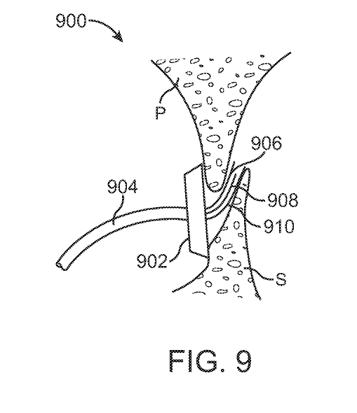

In an alternative embodiment, RF energy may be applied to the tunnel of the PFO or between the septum primum and septum secundum tissue layers. In FIG. 9, another multipolar electrosurgical catheter 900 is shown. In FIG. 9, the multipolar electrosurgical catheter 900 includes a catheter shaft 904 with a resilient housing 902 coupled to the distal end of the catheter shaft. Three electrodes 906, 908, 910 extend from the resilient housing 902 into the PFO, between tissue layers P, S. Electrodes 906 and 910 are electrically coupled together and electrode 908 is isolated from the other two electrodes 906, 910. The three electrodes are advanced from catheter shaft 904 into the PFO tunnel and an optional vacuum may be applied to help the resilient housing 902 and tissues P, S appose one another. RF energy is then applied to electrodes 906, 908 and 910 using the multipolar modality previously described to heat and fuse the PFO tissues P, S together, thereby closing the tissue defect. The electrodes 906, 908 and 910 may simultaneously be retracted during RF energy delivery, thus as the PFO tunnel seals, the electrodes 906, 908, 910 are retracted to prevent tissue from adhering to electrodes of the electrosurgical catheter 900. Other electrode configurations are possible and this embodiment is not intended to be limiting. For example, other exemplary electrode configurations for treating a PFO tunnel are disclosed in U.S. patent application Ser. No. 11/464,746 (Attorney Docket No. 022128-000301US) filed Aug. 15, 2006 and U.S. patent application Ser. No. 11/464,755 (Attorney Docket No. 022128-000208US) filed Aug. 15, 2006, the entire contents of which are hereby incorporated by reference.

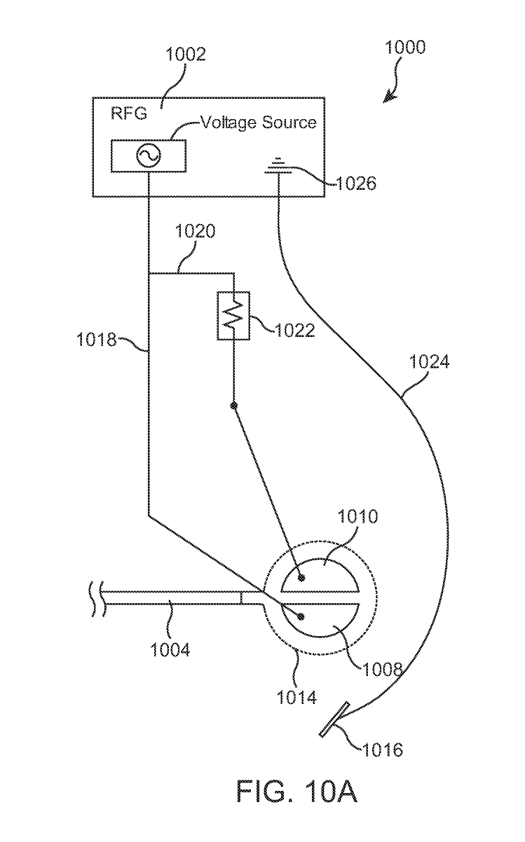

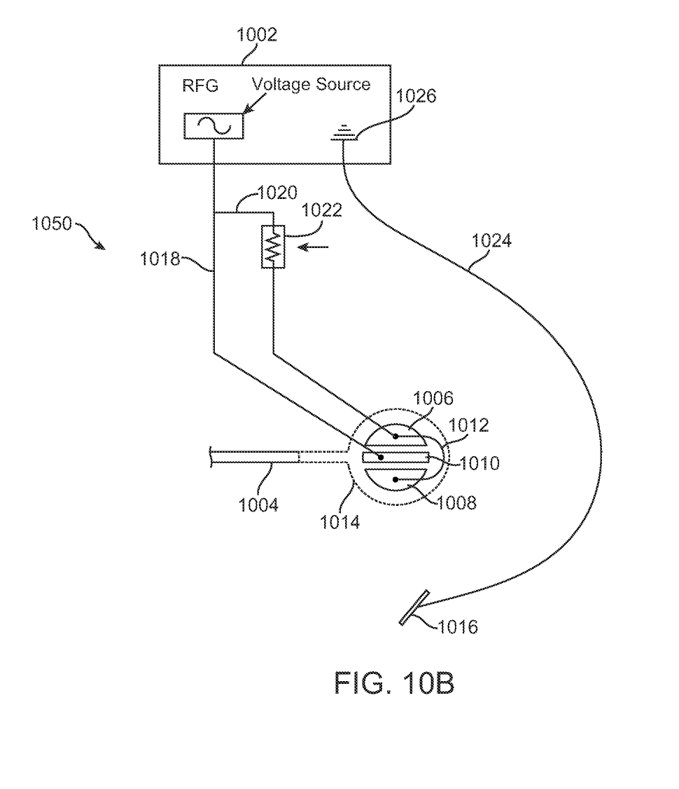

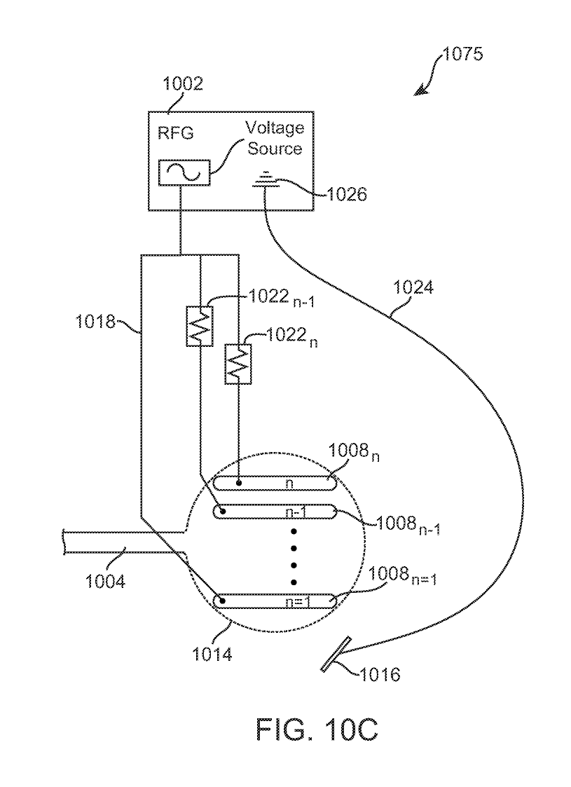

RF energy may be applied to the electrodes of a multipolar electrosurgical system in several different ways. For example, FIG. 10A shows a schematic diagram of how a resistor circuit 1022 may be used to create a difference in potential across the electrodes in a multipolar electrosurgical system. In FIG. 10A, a single power supply 1002 is used to deliver energy to the electrodes 1008, 1010 of a multipolar electrosurgical catheter system 1000. Catheter system 1000 comprises a RF power supply 1002 and a multipolar electrosurgical catheter 1004. The electrosurgical catheter 1004 includes a resilient housing 1014 at its distal end and two electrodes 1008, 1010. Voltage is applied from the RF power supply 1002 via conductor 1018 to electrode 1008. Voltage is also supplied from RF power supply 1002 via conductor 1020, across resistor circuit 1022 in series with and to electrode 1010. Resistor circuit 1022 may have a resistor of fixed value or it may be a variable resistor and results in a lower potential being delivered to electrodes 1010 as compared to the potential delivered to electrode 1008. The value of resistor circuit 1022 may be adjusted to control the difference in potential between electrode 1008 and electrode 1010. Resistor circuit 1022 often has a resistance of between 5Ω and 100Ω, preferably between 5Ω and 50Ω, and more typically between 5Ω and 25Ω. It is important to note however, that resistance depends on the system impedance of the tissue being treated. With a voltage drop of 0% between electrodes 1008 and 1010, only monopolar current flow results, while on the other hand, when there is a 100% voltage drop between electrodes 1008 and 1010, bipolar current flow results. Thus, the resistor circuit 1022 may be used to control the degree of multipolar current flow, and at present it is believed that a voltage drop of approximately 10-20% between electrodes 1008 and 1010 works well, although higher or lower percentage voltage drops will also work. Resistance can therefore be adjusted to provide such a voltage drop. Therefore, in some embodiments, resistor circuit 1022 also includes a resistor control circuit that controls the resistance thereby controlling the path of current flow between the active electrodes. Because of the higher potential across electrodes 1008, 1010 relative to ground 1026, current will flow from electrodes 1008, 1010 to return electrode 1016 and back to the ground 1026 of RF power supply 1002 via conductor 1024 in a monopolar mode. Additionally, because the potential across electrode 1008 is higher relative to electrode 1010, current will flow between electrode 1010 and electrode 1008 in a quasi-bipolar mode. Again, the current flow is described as quasi-bipolar because the current does not flow directly from electrode 1008 back to the power supply 1002, but instead flows from electrode 1008 through the a patient's tissue to the return electrode 1016.

FIG. 10B shows a slight variation on the schematic diagram of FIG. 10A. In FIG. 10B, a single power supply 1002 is used to deliver energy to the electrodes 1006, 1008, 1010 of a multipolar electrosurgical catheter system 1050. Electrodes 1006 and 1008 are electrically coupled together by conductor 1012. Voltage is applied from the RF power supply 1002 via conductor 1018 to electrode 1010. Voltage is also supplied from RF power supply 1002 via conductor 1020, across resistor circuit 1022 in series with and to electrodes 1006 and 1008. Resistor circuit 1022 may have a fixed value or may be a variable resistor used to adjust the potential applied to electrodes 1006 and 1008, and results in a lower potential being delivered to electrodes 1006 and 1008 as compared to the potential delivered to electrode 1010. A resistor control circuit, as described above with respect to 1022 in FIG. 10A may also be incorporated into this embodiment. Because of the higher potential across electrodes 1006, 1008, 1010 relative to ground 1026, current will flow from electrodes 1006, 1008, 1010 to return electrode 1016 and back to the ground 1026 of RF power supply 1002 via conductor 1024 in a monopolar mode. Additionally, because the potential across electrode 1010 is higher relative to electrodes 1006 and 1008, current will flow from electrode 1010 to both electrodes 1006 and 1008 in a quasi-bipolar mode. Hence, in this embodiment, both monopolar and quasi-bipolar modalities are used to deliver RF energy to tissues in order heat up and weld them together.

FIG. 10C shows another slight variation on the schematic diagram of FIG. 10A. In FIG. 10C, a single power supply 1002 is used to deliver energy to the n-number electrodes 1008ₙ of a multipolar electrosurgical catheter system 1075. One or more resistor circuits 1022ₙ are provided in series with the electrodes 1008ₙ such that the potential on at least one electrode 1008ₙ is different from the potential at another electrode 1008ₙ. The resistor circuits 1022ₙ may be a fixed value or they may be variable resistors so that the applied potential can be adjusted and they may include the resistor control circuit previously discussed above with reference to 1022 in FIG. 10A. If desired, n−1 resistor circuits 1022ₙ may be used with one resistor provided in series with each of n−1 electrodes such that the potential is different at each of the n-number of electrodes 1008ₙ. It is not necessary to provide a resistor in series with the nth electrode. Because of the higher potential across electrodes 1008₁, 1008₂, . . . , 1008ₙ₋₁, and 1008ₙ relative to ground 1026, current will flow from electrodes 1008₁, 1008₂, . . . , 1008ₙ₋₁, and 1008ₙ to return electrode 1016 and back to the ground 1026 of RF power supply 1002 via conductor 1024 in a monopolar mode. Additionally, because the potential across electrode 1008₁ is higher relative to electrodes 1008₂, . . . , 1008ₙ₋₁, and 1008ₙ current will flow from electrode 1008₁ to electrodes 1008₂, . . . , 1008ₙ₋₁, and 1008ₙ in a quasi-bipolar mode.

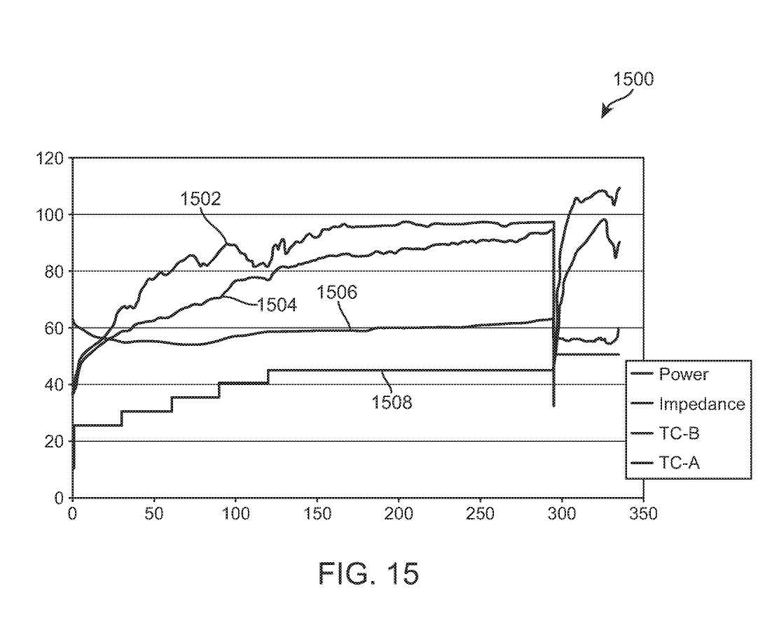

A device embodying the schematic of FIG. 10B was tested in vitro on porcine cardiac tissue having a PFO. In FIG. 15 a graph 1500 illustrates the relationship between power 1508, temperature 1502 of center electrode 1010, temperature 1504 of outer electrode 1006 and tissue impedance 1506. A 12Ω resistor was used. FIG. 15 shows that the temperature 1502 of center electrode 1010 is consistently hotter than the temperature 1504 of outer electrode 1006 during the multipolar delivery of energy. This demonstrates that bipolar current flow exists and current is directed toward the center electrode 1010, as opposed to simple monopolar energy delivery where current would tend to flow to the outer electrodes thereby resulting in a cooler center electrode 1010.

The multipolar method described above was used to weld porcine PFOs closed. Data collected included size of the PFO, volume of blood loss and the leakage flow rate. Average temperature, average power, energy delivered and energy delivery times were also recorded along with the burst strength. Notes were also recorded during the testing such as the color of the tissue after treatment (e.g. pink) as well as the number of impedance spikes observed (e.g. 3 spikes). A 4 L/min saline flow was provided to the left atrium of the PFO. The quality of the seal was tested using burst pressure for several samples as summarized in Table 1 below. This data was then compared to data obtained from monopolar PFO closure using the methods described in U.S. patent application Ser. No. 11/403,052 (Attorney Docket No. 022128-000720US) which is summarized in Table 2 below. Average PFO burst pressure using the multipolar method described herein was higher than that obtained under monopolar conditions. For example, the average multipolar burst pressure was 100 mm Hg, ten times higher than the average of 10 mm Hg for monopolar. Likewise, the range of minimum and maximum burst pressures was also correspondingly higher for multipolar delivery (76 mm Hg to 200 mm Hg) than monopolar delivery (0 mm to 28 mm Hg). In addition to the higher burst pressures obtained using multipolar delivery, on average, lower power and energy were required in the multipolar modality (33.1 W and 10.5 kJ) than the monopolar modality (36.5 W and 18.4 kJ), indicating that the multipolar method is more efficient than the monopolar method. This is further evidenced by the lower time required to close the PFOs using multipolar versus monopolar (313 seconds versus 498 seconds, respectively). The data obtained from dynamic bench testing therefore show that the multipolar modality is a promising means for closing PFOs. It is important to note, however, that the data is for illustrative purposes only. Higher fluid flow (leak, etc.) may impact the amount of energy delivered and therefore the power.

TABLE 1
PFO Burst Test Results Using Multipolar RF Delivery.

| | PFO | | Av | Av | RF | | | | |
|---|---|---|---|---|---|---|---|---|---|
| | size | B.Loss | Temp | Power | Time | Leak | Energy | Failure | |
| # | (mm) | (ml) | (° C.) | (W) | (sec) | (ml/min) | (kJ) | (mmHg) | Notes |
| 2 | 7 | 100 | 67.3 | 32.5 | 252 | 24 | 8.2 | 88 | 3 spikes |
| 4 | 9 | 150 | 72.4 | 34.9 | 486 | 19 | 17.0 | 103 | 3 spikes |
| 6 | 8 | 200 | 66.4 | 32.9 | 254 | 47 | 8.4 | 76 | 3 spikes |
| 1 | 7 | 275 | 65.7 | 34.3 | 301 | 55 | 10.3 | 102 | 3 spikes |
| 2 | 7 | 200 | 50.3 | 36.4 | 407 | 29 | 14.8 | 77 | 3 spikes |
| 7 | 9 | 100 | 63.8 | 32.8 | 291 | 21 | 9.5 | 86 | 3 spikes |
| 8 | 9 | 0 | 63.5 | 31.2 | 228 | 0 | 7.1 | 78 | 3 spikes |
| 1 | 8 | 350 | 62.8 | 33.3 | 367 | 57 | 12.2 | 200 | did not |
| | | | | | | | | | burst, |
| | | | | | | | | | 3 spikes |
| 2 | 7 | 0 | 77.5 | 33.4 | 343 | 0 | 11.5 | 114 | 3 spikes |
| 3 | 7 | 0 | Not | 32.5 | 281 | 0 | 9.1 | 87 | 3 spikes |
| | | | Recorded | | | | | | |
| 4 | 6 | 0 | Not | 33.6 | 306 | 0 | 10.3 | 89 | 3 spikes |
| | | | Recorded | | | | | | |
| 5 | 9 | 0 | Not | 31.1 | 235 | 0 | 7.3 | 102 | 3 spikes |
| | | | Recorded | | | | | | |
| AVG | 7.8 | 115 | 65.5 | 33.2 | 313 | 21 | 10.5 | 100 | |
| Min | 6.0 | 0 | 50.3 | 31.1 | 228 | 0 | 7.1 | 76 | |
| Max | 9.0 | 350 | 77.5 | 36.4 | 486 | 57 | 17.0 | 200 | |

TABLE 2
PFO Burst Test Results Using Monopolar RF Delivery.

| | PFO | | Av | Av | RF | | | | |
|---|---|---|---|---|---|---|---|---|---|
| | size | B.Loss | Temp | Power | Time | Leak | Energy | Failure | |
| # | (mm) | (ml) | (° C.) | (W) | (sec) | (ml/min) | (kJ) | (mmHg) | Notes |
| 1 | n/a | 650 | 60.9 | 40.0 | 600 | 65 | 24.0 | 0 | Pink spot |
| | | | | | | | | | no spike |
| 3 | 8 | 400 | 68.3 | 33.5 | 461 | 52 | 15.4 | 0 | 3 spikes |
| | | | | | | | | | pink spot |
| 5 | 7 | 700 | 74.0 | 35.0 | 600 | 70 | 21.0 | 20 | pink spot |
| | | | | | | | | | no spike |
| 3 | 8 | 250 | 53.4 | 39.8 | 582 | 26 | 23.2 | 23 | 1 spike |
| | | | | | | | | | pink spot |
| 4 | 6 | 900 | 53.5 | 39.1 | 512 | 105 | 20.0 | 0 | 1 spike |
| | | | | | | | | | pink spot |
| 9 | 6 | 250 | 51.0 | 35.4 | 427 | 35 | 15.1 | 0 | 2 spikes |
| | | | | | | | | | pink spot |
| 10 | 7 | 0 | 66.0 | 33.0 | 303 | 0 | 10.0 | 28 | 3 spikes |
| Avg | 7.0 | 450 | 61.0 | 36.5 | 498 | 50 | 18.4 | 10 | |
| Min | 6.0 | 0 | 51.0 | 33.0 | 303 | 0 | 10.0 | 0 | |
| Max | 8.0 | 900 | 74.0 | 40.0 | 600 | 105 | 24.0 | 28 | |

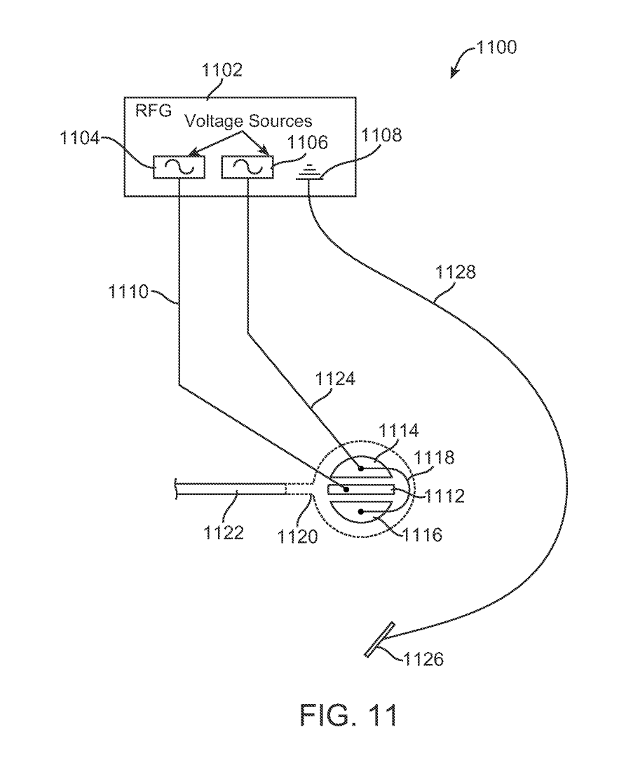

FIG. 11 shows the three electrode multipolar electrosurgical system 350 of FIG. 3B incorporated into a resilient housing disposed on the distal end of a catheter. In FIG. 11, RF power supply 1102 includes two power supplies 1104 and 1106, with power supply 1104 delivering a higher potential by conductor 1110 to electrode 1112 relative to power supply 1106. Power supply 1106 delivers the lower potential RF energy by way of conductor 1124 to electrodes 1114 and 1116 which are coupled together by conductor 1118 so that they are both at the same potential. Electrodes 1112, 1114 and 1116 are coupled to a resilient housing 1120 which is disposed on the distal end of catheter shaft 1122. As previously described above, because the potentials across electrodes 1112, 1114 and 1116 are higher than ground, current flows from electrodes 1112, 1114, 1116 to return electrode 1126 and back to the ground 1108 of the RF power supply 1102 via conductor 1128. Additionally, current flows from electrode 1112 to both electrodes 1114 and 1116 because the potential across electrode 1112 is higher relative to electrodes 1114 and 1116 and current also flows from electrodes 1114 and 1116 back to the power supply 1106. One skilled in the art would also recognize that two electrode embodiment of system 300 in FIG. 3A and the N electrode embodiment of system 375 in FIG. 3C could also be incorporated into a resilient housing coupled to the distal end of a catheter shaft.

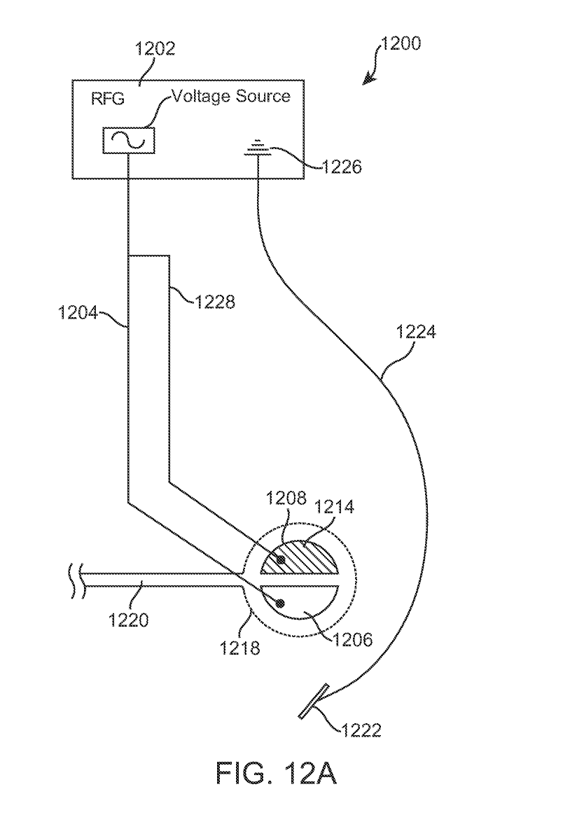

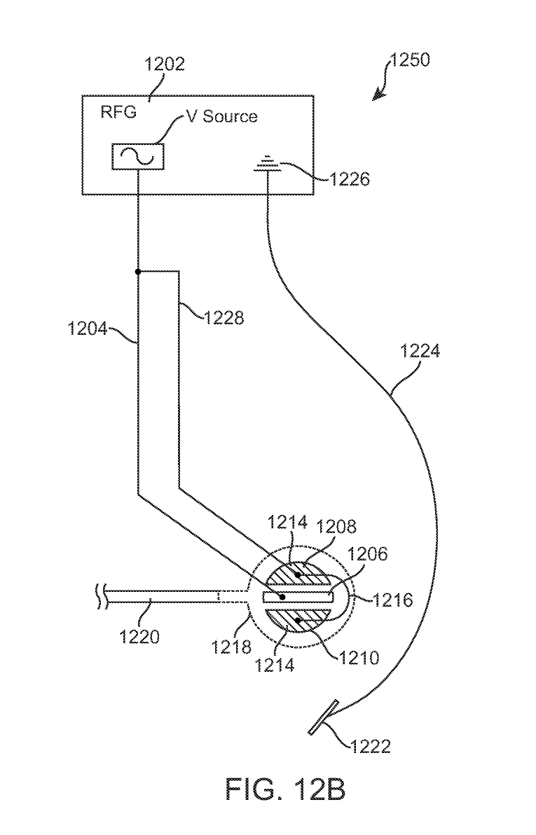

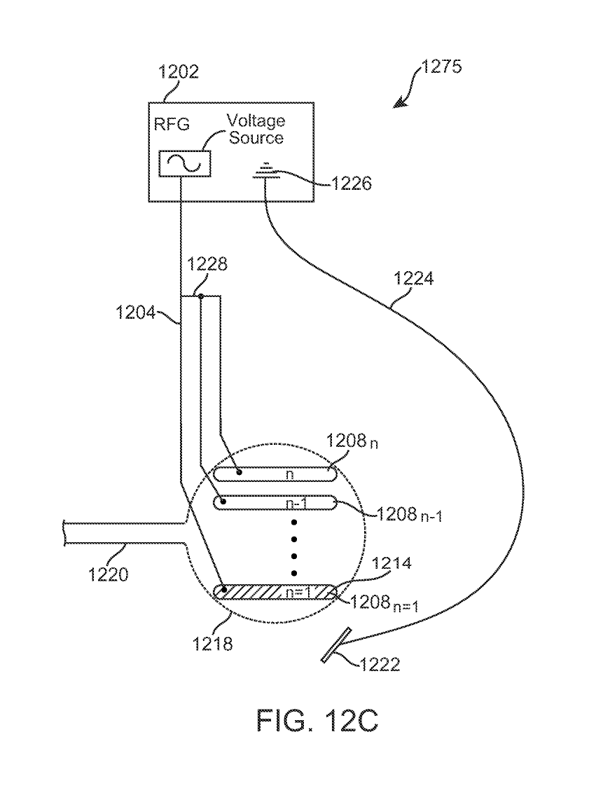

Another embodiment of a single RF power source is illustrated in FIG. 12A. In FIG. 12A, a single RF supply 1202 is used to deliver RF energy to electrodes 1206 and 1208 of multipolar electrosurgical system 1200. RF energy is delivered via conductor 1204 to electrode 1206. RF energy is also delivered by conductor 1228 to electrode 1208. Unlike the embodiments in FIGS. 10A and 10B which employ an inline resistor 1022 to create a potential difference, in this embodiment electrode 1208 is fabricated from a material that has a higher resistance than typically found in conductive materials, such as nichrome or graphite. Therefore, electrodes 1208 acts as if a resistor such as resistor 1022 in FIGS. 10A and 10B were placed in the circuit resulting in a lower potential being delivered to electrode 1208. Optionally, a resistive coating such as graphite 1214 or other similar material may be applied to the surface of electrode 1208 to create the higher resistance. Similar to previous embodiments in FIGS. 10A and 11, electrodes 1206 and 1208 are coupled to a resilient housing 1218 disposed on the distal end of a catheter shaft 1220. Current then flows in a monopolar fashion from electrodes 1206 and 1208 to return electrode 1222 and back to the ground 1226 of RF power supply 1202 via conductor 1224. Additionally, current flows in a quasi-bipolar manner between electrode 1206 and electrode 1208. Another embodiment of a single RF power source is illustrated in FIG. 12B. In FIG. 12B, a single RF supply 1202 is used to deliver RF energy to electrodes 1206, 1208, 1210 of a multipolar electrosurgical system 1250. Electrodes 1208 and 1210 are manufactured from a higher resistance material such as nichrome or graphite or may be coated with a higher resistance material 1214 such as graphite in order to increase their resistance, so that a lower potential is delivered to electrodes 1208 and 1210 relative to electrode 1206. RF energy is delivered via conductor 1204 to electrode 1206. RF energy is also delivered by conductor 1228 to electrodes 1208 and 1210 which are electrically coupled together by conductor 1216.

In FIG. 12B, electrodes 1206, 1208, 1210 are coupled to a resilient housing 1218 disposed on the distal end of a catheter shaft 1220. Current flows in a monopolar fashion from electrodes 1206, 1208 and 1210 to return electrode 1222 and back to the ground 1226 of RF power supply 1202 via conductor 1224. Additionally, current flows in a quasi-bipolar manner between electrode 1206 and electrodes 1208, 1210.

Another embodiment of a single RF power source is illustrated in FIG. 12C. In FIG. 12C, a single RF supply 1202 is used to deliver RF energy to a plurality of electrodes 1208₁, 1208₂, 1208₃, . . . , 1208ₙ of a multipolar electrosurgical system 1275. At least one of the electrodes 1208₁ is fabricated from a material that has a higher resistance than typically found in conductive materials, such as nichrome or graphite. In this manner, the potential is different at one electrode 1208₁ relative to one or more of the remaining electrodes 1208₂, 1208₃, . . . , 1208ₙ, thereby producing monopolar and quasi-bipolar current flow.

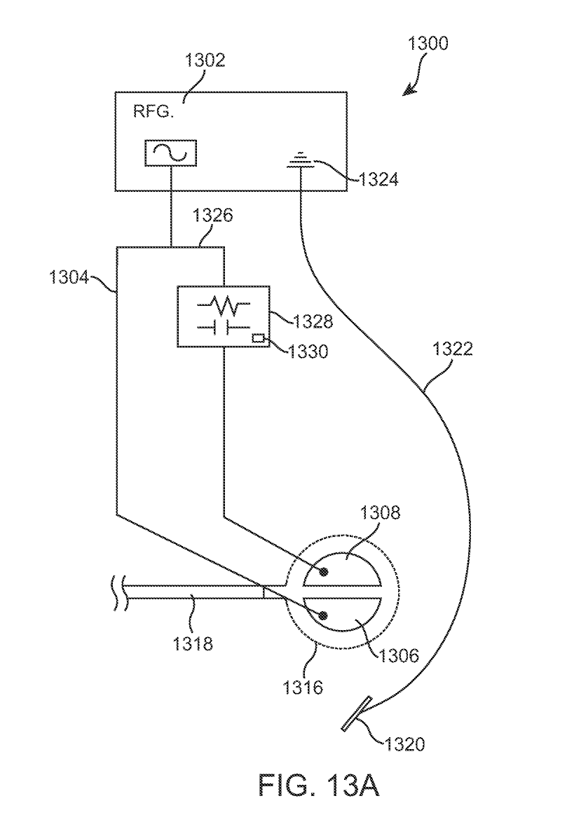

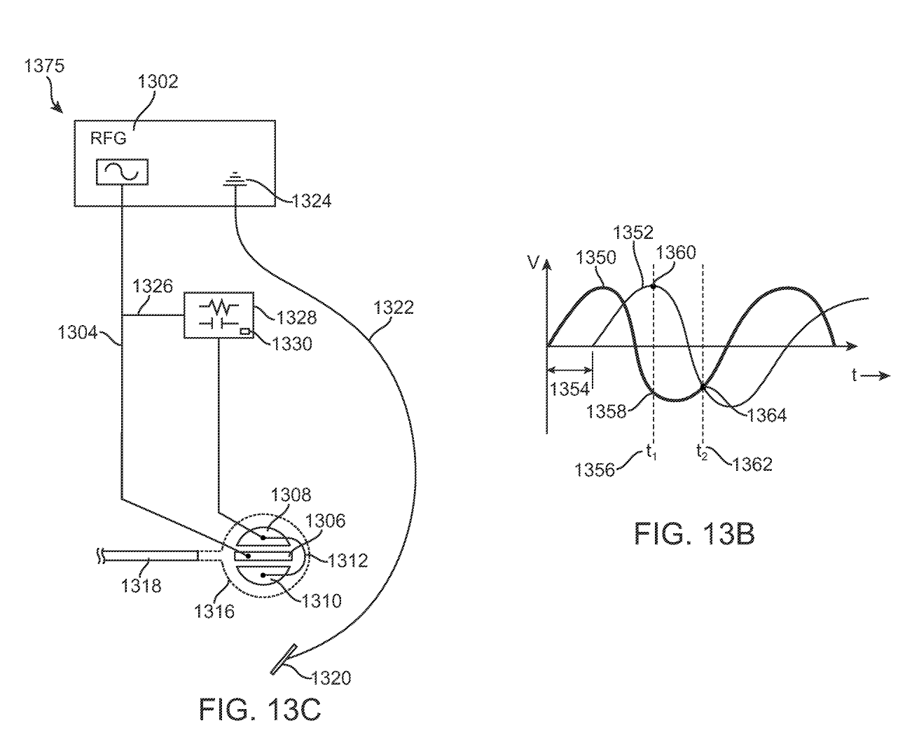

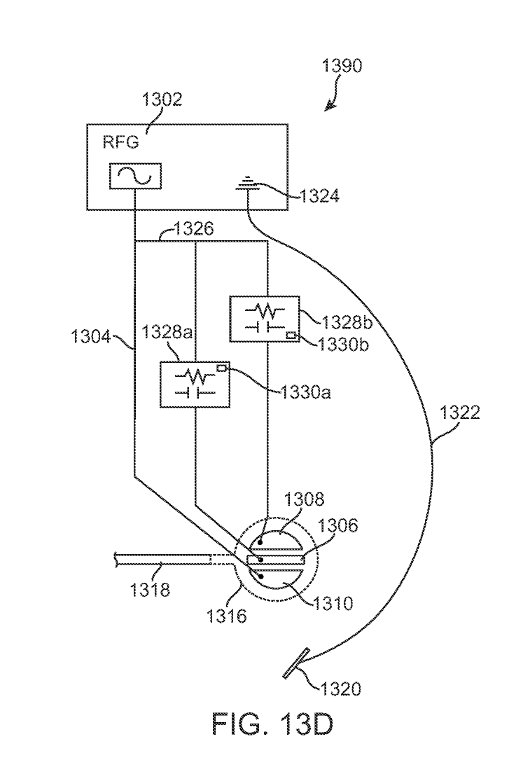

The prior embodiments rely upon controlling amplitude to create two different potentials across the electrodes of the multipolar electrosurgical system. Phase control may also be used to deliver different potentials of RF energy to the electrodes as seen in FIGS. 13A-13C. In FIG. 13A a multipolar, phase controlled electrosurgical system comprises a RF power supply 1302, electrodes 1306 and 1308. Electrodes 1306 and 1308 are coupled to a resilient housing 1316 attached to the distal end of a catheter shaft 1318.

In FIG. 13A, a single RF power supply 1302 is used in the phase controlled multipolar electrosurgical system 1300. Power supply 1302 delivers RF energy via conductor 1304 to electrode 1306. The RF energy delivered along conductor 1304 has a defined waveform 1350 as seen in FIG. 13B. RF energy is also delivered from power supply 1302 along conductor 1326 through a resistor-capacitor (RC) circuit 1328 to electrode 1308. The RC circuit 1328 causes a phase shift in the waveform 1360 of RF energy delivered to electrode 1308 as seen in FIG. 13B. Waveform 1360 has the same frequency and amplitude as waveform 1350 with the exception that it is shifted out of phase by an amount 1354 determined by the time constant τ of RC circuit 1328. Phase shifting circuits are well known in the art and widely reported in the scientific and patent literature. The RC circuit 1328 may also include a control circuit 1330 that controls operation of the power supply 1302 and the RC circuit 1328 so as to vary the amount of current traveling from one electrode to another electrode. Thus current flow could be varied over time. Additionally, the RC control circuit 1330 could vary the RC time constant in response to a measured tissue impedance or temperature value so as to vary the current flow between electrodes.

Shifting the phase of the RF energy delivered to electrode 1308 results in a different potential delivered to electrode 1308 as compared to the potential delivered to electrode 1306. For example, as illustrated in FIG. 13B, at time t₁ 1356, the amplitude 1360 of waveform 1352 exceeds the amplitude 1358 of waveform 1350. Thus, a higher potential would be delivered to electrode 1308 relative to the potential delivered to electrode 1306. At other times, the potential from waveform 1350 is higher than the potential from waveform 1352 and thus the potential delivered to electrode 1306 exceeds that delivered to electrode 1308. Still, at other times, when the two waveforms 1350, 1352 cross each other, for example at time t₂ 1362, the amplitude of both waveforms 1350 and 1352 is the same and therefore potential across all electrodes 1306 and 1308 are equal. Whenever the potential between electrodes 1306 and 1308 differ, quasi-bipolar conduction occurs, between electrodes 1306 and 1308. Current also flows in a monopolar modality from electrodes 1306 and 1308 to return electrode 1320 and back to the ground 1324 of power supply 1302 by conductor 1322. When the potential across electrodes 1306 and 1308 is equal, there will be no quasi-bipolar current flow, however, current will still flow in a monopolar fashion back to return electrode 1320 and RF power supply 1302 ground 1324 via conductor 1322.

FIG. 13C depicts a slight variation of the system of FIG. 13A including electrodes 1308 and 1310 coupled together by conductor 1312 in system 1375. All three electrodes 1306, 1308, 1310 are coupled to a resilient housing 1316 attached to the distal end of a catheter shaft 1318. RF energy is also delivered from power supply 1302 along conductor 1326 through a resistor-capacitor (RC) circuit 1328 to electrodes 1308 and 1310 which are coupled together by conductor 1312. The RC circuit 1328 causes a phase shift in the waveform 1360 of RF energy delivered to conductors 1308 and 1310 as seen in FIG. 13B. The waveform 1360 has the same frequency and amplitude as waveform 1350 with the exception that it is shifted out of phase by an amount 1354 determined by RC circuit 1328. RC circuit 1328 may include the RC control circuit 1330 previously described in FIG. 13A above.

Shifting the phase of the RF energy delivered to the second group of electrodes, 1308, 1310, results in a different potential delivered to electrodes 1308, 1310 as compared to the potential delivered to electrode 1306. For example, as illustrated in FIG. 13B, at time t₁ 1356, the amplitude 1360 of waveform 1352 exceeds the amplitude 1358 of waveform 1350. Thus, a higher potential would be delivered to electrodes 1308 and 1310 relative to the potential delivered to electrodes 1306. At other times, the potential from waveform 1350 would be higher than the potential from waveform 1352 and thus the potential delivered to electrode 1306 exceeds that delivered to electrodes 1308, 1310. Still, at other times, when the two waveforms 1350, 1352 cross each other, for example at time t₂ 1362, the amplitude of both waveforms 1350 and 1352 is the same and therefore potential across all three electrodes 1306, 1308 and 1310 would be equal. Whenever the potential between electrodes 1306, 1308 and 1310 differ, quasi-bipolar conduction occurs, either from electrode 1306 to electrodes 1308 and 1310, or from electrodes 1308, 1310 to 1306. Current also flows in a monopolar modality from electrodes 1306, 1308 and 1310 to return electrode 1320 and back to the ground 1324 of power supply 1302 by conductor 1322. When the potential across all three electrodes 1306, 1308, 1310 is equal, there will be no quasi-bipolar current flow, however, current will still flow in a monopolar fashion back to return electrode 1320 and RF power supply 1302 ground 1324 via conductor 1322.

FIG. 13D shows another variation on the phase shifting system 1375 of FIG. 13C including the use of multiple RC circuits to control the phase of the power delivered to different electrodes. The system 1390 in FIG. 13D comprises three electrodes 1306, 1308 and 1310 coupled to a resilient housing 1316 disposed on the distal end of a catheter shaft 1318. In FIG. 13D, RF energy is delivered to a electrode 1310 via conductor 1304. RF energy is also delivered via conductor 1326 through RC circuit 1328a to electrode 1306 and RF energy is delivered over conductor 1326 through RC circuit 1328b to electrode 1308. Either one or both RC circuits 1328a and 1328b may also include the RC control circuits 1330a and 1330b which generally take the same form as those previously described in FIG. 13A above. The values of the resistors and capacitors in RC circuits 1328a and 1328b may be fixed or variable in order to control the resulting phase shift of the RF energy applied to electrodes 1306 and 1308. Varying the phase shift will vary the potential differences between electrodes 1306, 1308 and 1310 thereby affecting the monopolar current flow from electrodes 1306, 1308 and 1310 to return electrode 1320 back to the ground 1324 of power supply 1302. Varying the phase shift will also affect the quasi-bipolar current flow between electrodes 1306, 1308 and 1310.

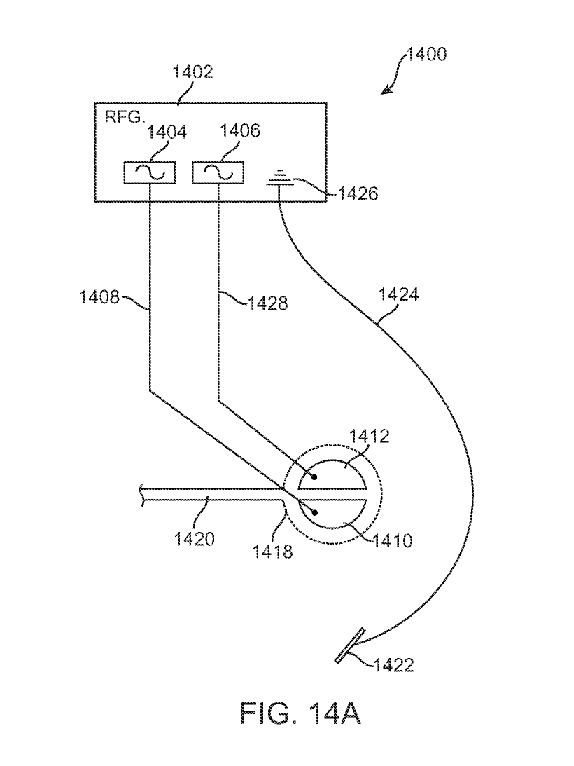

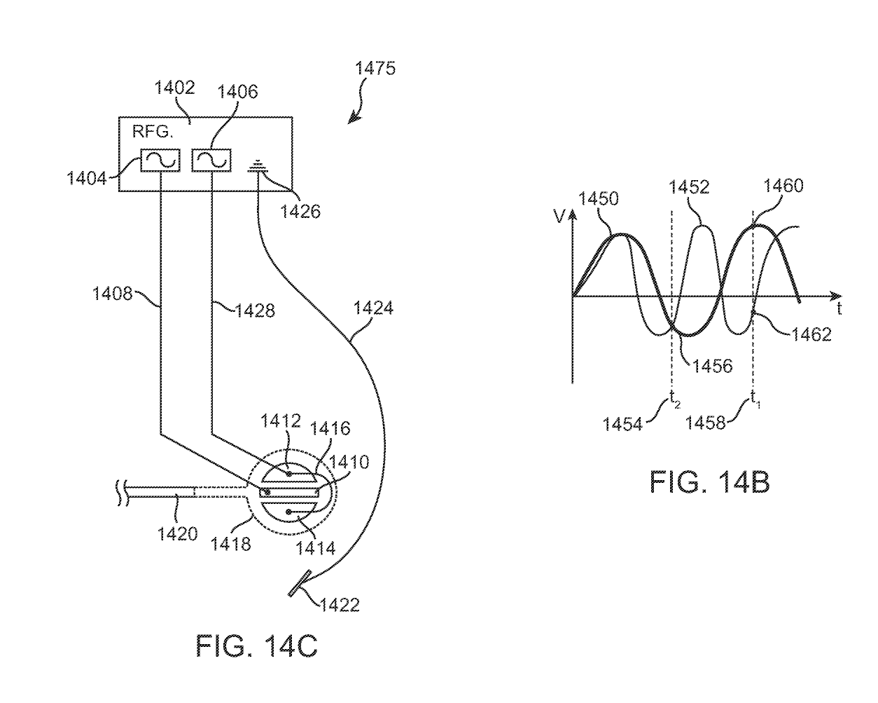

In addition to phase control, as discussed above, frequency control may also be used to deliver varying potentials to the electrodes of a multipolar electrosurgical system, such as in FIGS. 14A-14C. FIG. 14A is a schematic diagram of a frequency controlled multipolar electrosurgical system 1400 employing a RF power supply capable of providing power at two different frequencies 1404, 1406. In FIG. 14A, power from source 1404 is delivered via conductor 1408 to electrode 1410 at a first frequency 1450 seen in FIG. 14B. Power from a second supply 1406 at a second frequency 1452 is delivered along conductor 1428 to electrode 1412. Electrodes 1410 and 1412 are coupled to resilient housing 1418 disposed on the distal end of catheter shaft 1420.

Because two different frequencies 1450, 1452 of RF are delivered to electrodes 1410 and 1412, at any point in time, a different potential will generally be applied to the electrodes 1410 and 1412, as seen in FIG. 14B. In FIG. 14B for example, at time t₁ the amplitude 1460 of the first frequency 1450 wave is higher than the amplitude 1458 of the second frequency wave 1452. Therefore, a higher potential is delivered to electrode 1410 relative to the lower potential which is delivered to electrode 1412. At other times, the situation will be reversed and a lower potential is applied to electrode 1410 relative to electrode 1412, and still at other times, when the two waveforms cross each other, for example at time t₂, the amplitude of both waveforms is the same and hence the potential delivered to both electrodes 1410, 1412 is the same.

As long as there is a difference between potentials applied to electrode 1410 relative to electrode 1412, current will flow therebetween in a quasi-bipolar manner with additional monopolar current flow to return electrode 1422, through conductor 1424 back to ground 1426. When the potential applied to both electrodes 1410 and 1412 is the same, only classic monopolar current flow will result with current flowing from the electrodes 1410 and 1412 to return 1422 and back to the ground of RF power supply 1402 via conductor 1424.

FIG. 14C shows a variation of system 1400 shown in FIG. 14A. System 1475 in FIG. 14C includes electrodes 1412 and 1414 coupled together by conductor 1416. FIG. 14C is a schematic diagram of a frequency controlled multipolar electrosurgical system 1475 employing a RF power supply capable of providing power at two different frequencies 1404, 1406. In FIG. 14C, power from source 1404 is delivered via conductor 1408 to electrode 1410 at a first frequency 1450 seen in FIG. 14B. Power from a second supply 1406 at a second frequency 1452 is delivered along conductor 1428 to electrodes 1412 and 1414 which are coupled together by conductor 1416. All three electrodes 1410, 1412, 1416 are coupled to a resilient housing 1418 disposed on the distal end of catheter shaft 1420.

Because two different frequencies 1450, 1452 of RF are delivered to the electrodes 1410, 1412 and 1414, at any point in time, a different potential will generally be applied to the electrodes 1410, 1412 and 1414 as seen in FIG. 14B. In FIG. 14B for example, at time t₁ the amplitude 1460 of the first frequency 1450 wave is higher than the amplitude 1458 of the second frequency wave 1452. Therefore, a higher potential is delivered to electrode 1410 relative to the lower potential which is delivered to electrodes 1412 and 1414. At other times, the situation will be reversed and a lower potential is applied to electrode 1410 relative to electrodes 1412 and 1414, and still at other times, when the two waveforms cross each other, for example at time t₂, the amplitude of both waveforms is the same and hence the potential delivered to all three electrodes 1410, 1412, 1414 is the same.

As long as there is a difference between potentials applied to electrode 1410 relative to electrodes 1412 and 1414, current will flow therebetween in a quasi-bipolar manner with additional monopolar current flow to return electrode 1422, through conductor 1424 back to ground 1426. When potential applied to all three electrodes 1410, 1412, 1414 is the same, only classic monopolar current flow will result with current flowing from the electrodes 1410, 1412, 1414 to return 1422 and back to the ground of RF power supply 1402 via conductor 1424.

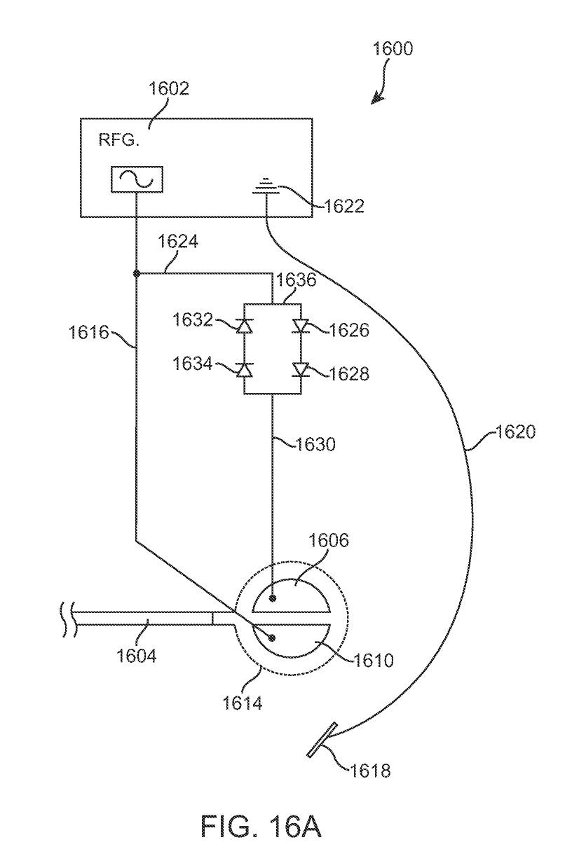

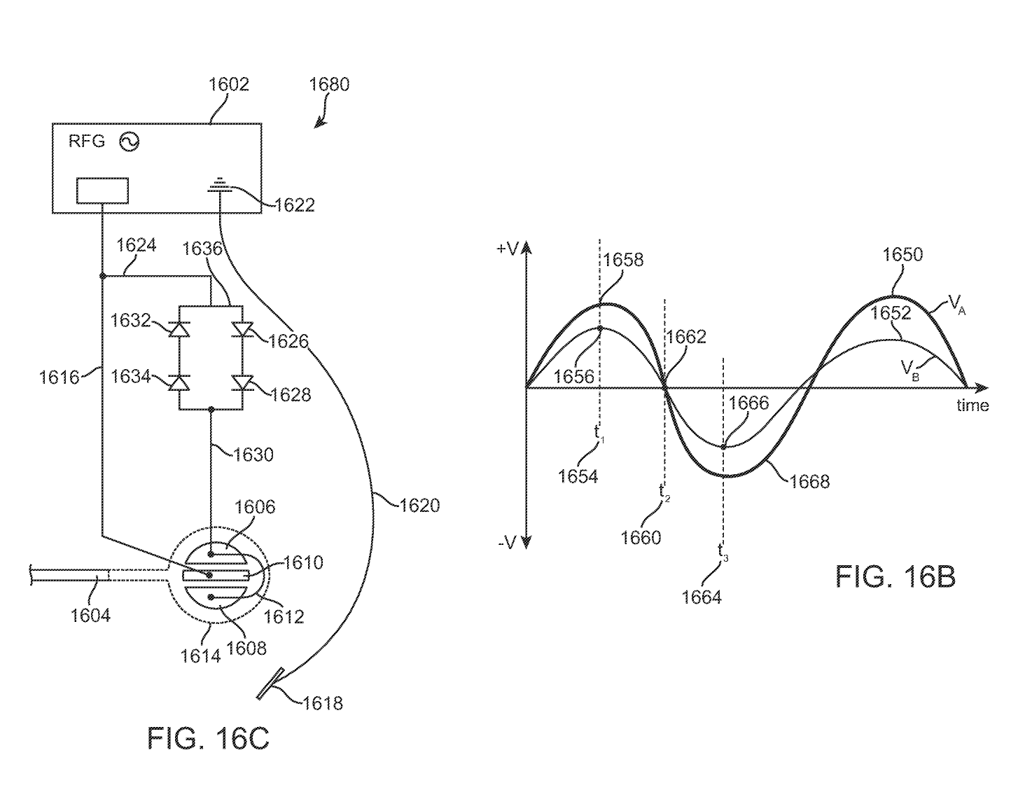

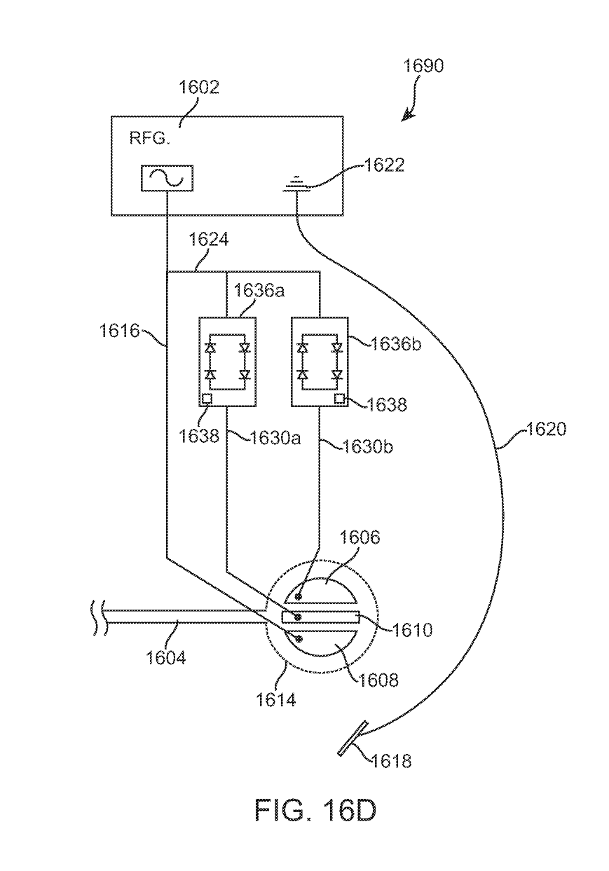

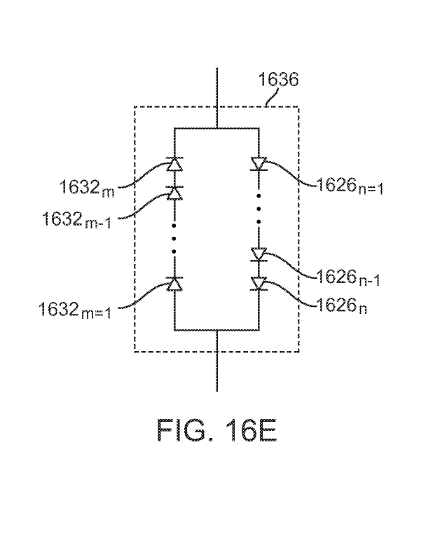

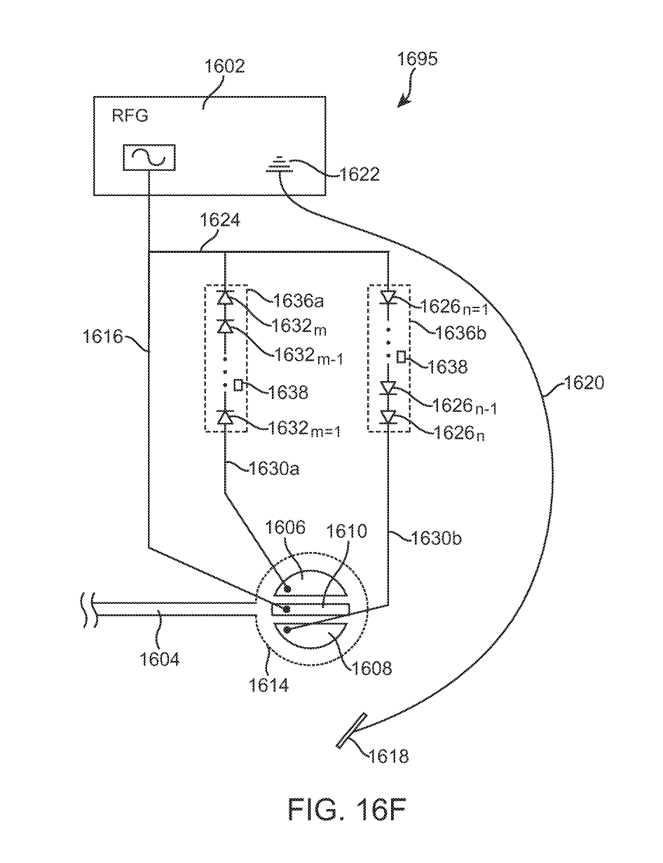

FIGS. 16A-16D are schematic diagrams of other embodiments of the present invention utilizing diodes that result in different potentials of RF energy being supplied to the system electrodes, thereby resulting in multipolar energy delivery. In FIG. 16A, a multipolar diode controlled electrosurgical system comprises a RF power supply 1602, electrode 1606 and electrode 1610. Electrodes 1606 and 1610 are coupled to a resilient housing 1614 attached to the distal end of a catheter shaft 1604.

In FIG. 16A, a single power supply, here an RF power supply 1602 is used in the diode controlled multipolar electrosurgical system 1600. RF power supply 1602 delivers RF energy via conductor 1616 to electrode 1610. The RF energy delivered along conductor 1616 has a defined waveform 1650 as seen in FIG. 16B. RF energy is also delivered from power supply 1602 along conductor 1624 through a diode circuit 1636 to electrode 1606 via conductor 1630. The diode circuit 1636 attenuates the voltage applied to electrode 1606. During a positive half cycle of RF energy, diodes 1626, 1628 allow current to flow toward electrode 1606, while diodes 1632, 1634 allow current to flow to electrode 1606 during the negative half of the cycle. Inherent properties of diodes however, result in a voltage drop across the diode. In typical silicon diodes, this voltage drop is typically around 0.6 to 0.7 Volts for each diode. Thus, in the exemplary diode circuit 1636, the voltage applied to electrode 1606 would be about 1.2 to 1.4 V lower, because of the two diodes in series, than the voltage applied to electrode 1608. The voltage drop is different in other diode types and can range from a low of about 0.2 V in a Schottky diode to 1.4 V for light emitting diodes (LED) and as high as 4 V in Blue LEDs. Thus, by using different quantities and different types of diodes in the diode circuit 1636, the voltage drop across the diode circuit 1636 may be adjusted, and hence there is a voltage drop across electrodes 1606 and 1610. FIG. 16E illustrates a diode circuit 1636 employing multiple diodes in series or “stacked” together in order to control the voltage drop across the diode circuit. In FIG. 16E, diode circuit 1636 includes N diodes 1626ₙ₌₁, . . . , 1626ₙ₋₁, 1626ₙ that attenuate the voltage on one half of the power cycle and M diodes 1632ₘ₌₁, . . . , 1632ₘ₋₁, 1632ₘ that attenuate the voltage on the other half of the power cycle. Thus, the diode circuit 1635 in FIG. 16E may be employed in any of the diode embodiments disclosed herein.

The resulting waveform of RF applied to electrode 1606 will have the same basic phase and frequency as waveform 1650, but will be attenuated. Waveform 1652 in FIG. 16B is used merely to illustrate the decrease in amplitude of the RF waveform supplied to electrode 1606, and may not accurately depict the actual waveform.

FIG. 16B shows how the diodes of circuit 1636 result in a lower potential being delivered to electrode 1606. Waveform 1650 shows the potential, V_{A} supplied by RF power supply 1602 to electrode 1610. Waveform 1652 is the attenuated, lower potential, V_{B} supplied by RF power supply 1602 to electrode 1606. Because of the attenuation of potential, the amplitude of waveform 1650 is greater than the amplitude of waveform 1652. Thus, for example at time t₁ 1654, the amplitude 1658 of waveform 1650 exceeds the amplitude 1656 of waveform 1652. A higher potential is therefore delivered to electrode 1610 relative to the potential to electrode 1606. At other times, such as time t₃ 1664 the potential 1666 of waveform 1652 will be more positive than the potential 1668 of waveform 1652 so the potential delivered to electrode 1606 is higher than that of electrode 1610. At other times, when the two waveforms 1650, 1652 cross each other, for example at time t₂ 1654, the amplitude of both waveforms 1650, 1652 is the same and therefore potential across both electrodes 1606, 1610 is equal.

Quasi-bipolar conduction occurs whenever the potential between electrodes 1606, 1610 differs. As described above, when the potential between electrodes 1606 and 1610 differs, current flows, either from electrode 1606 to electrode 1610 or from electrode 1610 to electrode 1606. Current also flows in a monopolar fashion from electrodes 1606 and 1610 to return electrode 1618 back to ground 1622 of power supply 1602 along conductor 1620.

FIG. 16C depicts a variation of system 1600 in FIG. 16A including electrodes 1606 and 1608 on either side of electrode 1610. Electrodes 1606 and 1610 are coupled together by conductor 1612 so they are at the same potential. System 1680 in FIG. 16C includes three electrodes 1606, 1608, 1610 coupled to a resilient housing 1614 attached to the distal end of a catheter shaft 1604. RF energy is also delivered from power supply 1602 along a conductor 1624 through a diode circuit 1636 to electrodes 1606, 1608 which are coupled together by conductor 1612. The diode circuit 1636 results in an attenuated RF voltage being applied to both electrodes 1606 and 1608 as discussed above with respect to FIGS. 16A-B. The number of diodes may be varied to control the attenuation as discussed with respect to FIG. 16E. Thus waveform 1650 shows the potential, V_{A} supplied by RF power supply 1602 to electrode 1610. Waveform 1652 is the attenuated, lower potential, V_{B} supplied by RF power supply 1602 to electrodes 1606, 1608. Because of the attenuation of potential, the amplitude of waveform 1650 is greater than the amplitude of waveform 1652. Thus, for example at time t₁ 1654, the amplitude 1658 of waveform 1650 exceeds the amplitude 1656 of waveform 1652. A higher potential is therefore delivered to electrode 1608 relative to the potential to electrode 1308. At other times, such as time t₃ 1664 the potential 1666 of waveform 1652 will be more positive than the potential 1668 of waveform 1652 so the magnitude of potential delivered to electrode 1606 is higher than that of electrode 1608. At other times, when the two waveforms 1650, 1652 cross each other, for example at time t₂ 1654, the amplitude of both waveforms 1650, 1652 is the same and therefore potential across both electrodes 1606, 1608 is equal.

Whenever the potential between electrodes 1606 and 1608 differs from that of electrode 1610, quasi-bipolar conduction occurs, either from electrodes 1606, 1608 to electrode 1610 or from electrode 1610 to electrodes 1606, 1608. Current also flows in a monopolar fashion from electrodes 1606, 1608 and 1610 to return electrode 1618 back to ground 1622 of power supply 1602 along conductor 1620.

FIG. 16D shows another variation on the diode embodiment of 1680 of FIG. 16C including the use of multiple diode circuits to control the potential delivered to different electrodes. System 1690 in FIG. 16D comprises three electrodes 1606, 1608 and 1610 coupled to a resilient housing 1614 disposed on the distal end of catheter shaft 1604. RF energy is delivered from power supply 1602 along conductor 1616 to electrode 1608. Power is also delivered along conductive path 1624 to diode circuit 1636a where potential drops as described above, then along conductor 1630a to electrode 1610. Similarly, power is delivered along conductor 1624 through diode circuit 1636b where potential drops and then along conductor 1630b to electrode 1606. The number of diodes in diode circuits 1636a and 1636b may also varied as discussed above in reference to FIG. 16E. Because electrodes 1606, 1608 and 1610 have different voltage drops across, monopolar and quasi-bipolar current flow will result, with some current returning to ground 1622 of power supply 1602 along conductor 1620. Additionally, diode circuits 1636a and 1636b may also include a diode control circuit 1638 that can adjust the diode circuit 1636a and/or 1636b so as to control the path that current flows between the active electrodes. This diode control circuit 1638 may also be employed in the embodiments described above in FIGS. 16A, 16C and 16F.

FIG. 16F shows a variation on the diode embodiment of 1690 of FIG. 16D, with the diode circuit being modified so that only half of the power cycle is delivered and attenuated to each of two electrodes surrounding a central electrode receiving the entire unattenuated power cycle. System 1695 in FIG. 16F comprises three electrodes 1606, 1608 and 1610 coupled to a resilient housing 1614 on the distal end of a catheter shaft 1604. RF energy is delivered from power supply 1602 along conductor 1616 to electrode 1610. Power is also delivered along conductive path 1624 to diode circuit 1636a where potential drops as described previously, but for only half the power cycle. The other half of the power cycle is cutoff due to the directionality of the M diodes 1632ₘ, 1632ₘ₋₁, . . . , 1632ₘ₌₁. As discussed above, the number of diodes M may be varied in order to obtain the desired voltage drop across diode circuit 1636a. Current then flows along conductor 1630a to electrode 1606. Similarly, power is delivered along conductor 1624 through diode circuit 1636b where potential drops for the other half of the power cycle and then current flows along conductor 1630b to electrode 1608. Again, the number of diodes N in diode circuit 1636b may be varied to obtain a desired voltage drop across the diode circuit 1636b.

Because electrodes 1606, 1608 and 1610 have different voltage drops, monopolar and quasi-bipolar current flow will result, with some current returning to ground 1622 of power supply 1602 along conductor 1620. Additionally, diode circuits 1636a and 1636b may also include a diode control circuit 1638 that can adjust the diode circuit 1636a and/or 1636b so as to control the path that current flows between the active electrodes.

Although the foregoing description is complete and accurate, it has described only exemplary embodiments of the invention. Various changes, additions, deletions and the like may be made to one or more embodiments of the invention without departing from the scope of the invention. Additionally, different elements of the invention could be combined (e.g. multiple amplitudes or multiple phases) to achieve any of the effects described above. Thus, the description above is provided for exemplary purposes only and should not be interpreted to limit the scope of the invention as set forth in the following claims.

What is claimed is:

1. A tissue coagulation system comprising:
a power source;
a plurality of active electrodes connected in parallel to said power source;
at least one resistor connected in series with one of said plurality of active electrodes such that the voltage drop across one of said active electrodes is different from the voltage drop across another of said active electrodes; and
a ground electrode,
wherein said power source is electrically coupled to said ground electrode through the tissue.

2. The system of claim 1, wherein said active electrodes are mounted to a resilient housing.

3. The system of claim 1, further comprising an impedance measuring circuit operably connected to said power source, said impedance measuring circuit measuring the impedance of the tissue.

4. The system of claim 1, further comprising at least one thermocouple mounted on at least one of said active electrodes.

5. The system of claim 2, further comprising at least one thermocouple mounted to said housing.

6. The system of claim 1, wherein a surface area of one of said active electrodes is larger than a surface area of another of said electrodes.

7. The system of claim 6, wherein said plurality of active electrodes comprise two active electrodes with one active electrode having a surface area at least three times as large as the surface area of the other active electrode.

8. The system of claim 7, wherein said plurality of active electrodes comprise two active electrodes with one of said two active electrodes comprising two segments which are adjacent to the other said two active electrodes.

9. The system of claim 1, wherein said ground electrode is generally remote from said active electrodes.

10. The system of claim 1, wherein adjacent ones of said electrodes are generally electrically insulated from one another such that current traveling between electrodes generally passes through tissue.

11. The system of claim 1, further comprising:
a catheter sized to fit within the vascular system of a mammal, said catheter having an elongate tubular housing;
wherein said active electrodes are housed within said elongate tubular housing in an undeployed state.

12. The system of claim 1, wherein an amount of current from said power source travels from one said active electrode through the tissue to another said active electrode and then through the tissue to said ground electrode, and another amount of current from said power source travels directly from one said active electrode through the tissue to said ground electrode.

13. The system of claim 1, further comprising a circuit controlling operation of said power source.

14. The system of claim 3, further comprising:
a control circuit operably coupled to said impedance measuring circuit and controlling operation of said power source;
wherein said control circuit discontinues the flow of power to said active electrodes when impedance measured by said impedance measuring circuit exceeds a threshold value.

15. The system of claim 14, wherein said impedance sets the threshold value to equal an initially measured value, initiates a flow of power to said active electrodes, and discontinues the flow of power to said active electrodes when impedance measured by said impedance measuring circuit exceeds said threshold value.

16. The system of claim 15, wherein said control circuit iterates through at least two power cycles where the control circuit sets the threshold value as an impedance value measured at the beginning of each said power cycle, initiates a flow of power to said active electrodes, and discontinues power for a predetermined rest period when an impedance value measured by said impedance measuring circuit exceeds the threshold impedance value stored at the beginning of that power cycle.

17. The system of claim 16, wherein the control circuit discontinues power and terminates iteration through any further power cycles once power has been applied for a predefined duration regardless of an impedance value measured by said impedance measuring circuit.

18. The system of claim 1, wherein said at least one resistor is a variable resistor.

19. The system of claim 18, further comprising a resistor control circuit controlling said at least one variable resistor to control the path of the current flow between said at least two active electrodes.

20. The system of claim 14, wherein:
said plurality of active electrodes comprises N-number of active electrodes;
said at least one resistor comprises N-number of variable resistors, with one of said variable resistors connected in series with each of said N-number of active electrodes; and
said control circuit controls resistance of said variable resistors so as to control the relative flow of current between said active electrodes.

21. The system of claim 20, wherein said control circuit includes a resistor control circuit controlling said plurality of variable resistors to control the path of the current flow between said active electrodes.

22. The system of claim 21, wherein said control circuit discontinues the flow of power to said active electrodes when impedance measured by said impedance measuring circuit exceeds a threshold value.

23. The system of claim 22, wherein said impedance measuring circuit measures an initial impedance of the tissue, and said control circuit discontinues the flow of power to said active electrodes when impedance measured by said impedance measuring circuit exceeds said initial impedance.

24. The system of claim 23, wherein said control circuit iterates through at least two power cycles, said control circuit stores an impedance value measured at the beginning of each said power cycle, applies power to said active electrodes, and discontinues power to said active electrodes for a predetermined rest period when an impedance value measured by said impedance measuring circuit exceeds the impedance value stored at the beginning of that power cycle.

25. The system of claim 24, wherein the control circuit discontinues power and terminates iteration through any further power cycles one power has been applied for a predefined duration regardless of an impedance value measured by said impedance measuring circuit.

26. A tissue coagulation welding system comprising:
a plurality of active electrodes; and
a plurality of power sources, with one said power source electrically coupled to one each of said active electrodes such that the voltage drop across one said active electrode is different from the voltage drop across a different of said active electrodes;
wherein each said power source is electrically coupled to a ground electrode through the tissue.

27. The system of claim 26, wherein said active electrodes are mounted to a resilient housing.

28. The system of claim 26, further comprising an impedance measuring circuit operably connected to at least one of said plurality of power sources, said impedance measuring circuit measuring the impedance of the tissue.

29. The system of claim 26, further comprising at least one thermocouple mounted on at least one of said at least two active electrodes.

30. The system of claim 27, further comprising at least one thermocouple mounted to said housing.

31. The system of claim 26, wherein a surface area of one of said active electrodes is larger than the surface area of another of said active electrodes.

32. The system of claim 26, wherein said plurality of active electrodes comprise two active electrodes with one active electrode having a surface area three times the surface area of the second active electrode.

33. The system of claim 26, wherein said plurality of active electrodes comprise first and second active electrodes, said second active electrode comprises two segments which are adjacent to said first active electrode.

34. The system of claim 26, wherein said ground electrode is generally remote from said at active electrodes.

35. The system of claim 26, wherein adjacent ones of said at least two active electrodes are electrically insulated from one another such that current traveling between electrodes generally passes through tissue.

36. The system of claim 26, further comprising:
a catheter sized to fit within the vascular system of a mammal, said catheter having an elongate tubular housing;
wherein said active electrodes are housed within said elongate tubular housing in an undeployed state.

37. The system of claim 26, wherein an amount of current from said plurality of power sources travels from one said active electrode through the tissue to another said active electrode and then through the tissue to said ground electrode, and another amount of current from said power sources travels directly from one said active electrode through the tissue to said ground electrode.

38. The system of claim 26, wherein an amount of current from said plurality of power sources travels from one said active electrode through the tissue to another said active electrode and then returns to said ground electrode, and another amount of current from said power sources travels directly from one said active electrode through the tissue to said ground electrode.

39. The system of claim 26, further comprising a control circuit controlling operation of said power sources.

40. The system of claim 28, further comprising:
a control circuit operably coupled to said impedance measuring circuit and controlling operation of said plurality of power sources;
wherein said control circuit discontinues the flow of power to said active electrodes when impedance measured by said impedance measuring circuit exceeds a threshold value.

41. The system of claim 40, wherein said impedance measuring circuit measures an initial impedance of the tissue, said control circuit sets the threshold value to equal said initial impedance, and said control circuit discontinues the flow of power to said at least two active electrodes when impedance measured by said impedance measuring circuit exceeds said initial impedance.

42. The system of claim 40, further comprising:
wherein said control circuit iterates through at least two power cycles where the control circuit stores an impedance value measured at the beginning of each said power cycle, applies power to said at least two active electrodes, and discontinues power for a predetermined rest period when an impedance value measured by said impedance measuring circuit exceeds the stored impedance value.

43. The system of claim 42, wherein the control circuit discontinues power and terminates iteration through any further power cycles once power has been applied for a predefined duration regardless of an impedance value measured by said impedance measuring circuit.

44. The system of claim 39, wherein said control circuit selectively controls power sources so as to vary the amount of current from traveling from one said active electrode to another said active electrode.

45. The system of claim 39, wherein said control circuit selectively controls said at least two power sources so as to vary over time the amount of current from traveling from one said active electrode to another said active electrode.

46. The system of claim 28, further comprising:
a circuit controlling operation of said plurality of power sources wherein said control circuit selectively controls said power sources so as to vary, in response to a detected impedance, the amount of current from traveling from one said active electrode to another said active electrode.

47. A tissue coagulation system comprising:
a power source;
a plurality of active electrodes connected in parallel to said power source,
wherein electrical characteristics of adjacent active electrodes are such that the voltage drop across one active electrode is different from the voltage drop across another active electrode; and
a ground electrode,
wherein said power source is electrically coupled to said ground electrode through the tissue.

48. The system of claim 47, wherein said active electrodes are mounted to a resilient housing.

49. The system of claim 47, further comprising an impedance measuring circuit operably connected to the power source, said impedance measuring circuit measuring the impedance of the tissue.

50. The system of claim 47, further comprising at least one thermocouple mounted on at least one of said plurality of active electrodes.

51. The system of claim 48, further comprising at least one thermocouple mounted to said housing.

52. The system of claim 47, wherein a surface area of one of said active electrodes is larger than a surface area of another said active electrode.

53. The system of claim 47, wherein said plurality of active electrodes comprise first and second active electrodes with said first active electrode having a surface area three times the surface area of the second active electrode.

54. The system of claim 53, wherein said plurality of active electrodes comprise first and second active electrodes with said second active electrode comprising two segments which are adjacent to said first active electrode.

55. The system of claim 47, further comprising:
a catheter sized to fit within the vascular system of a mammal, said catheter having an elongate tubular housing;
wherein said active electrodes are housed within said elongate tubular housing in an undeployed state.

56. The system of claim 47, wherein an amount of current from said power source travels from one said active electrode through the tissue to another said active electrode and then through the tissue to said ground electrode, and another amount of current from said power source travels directly from one said active electrode through the tissue to said ground electrode.

57. The system of claim 47, wherein an amount of current from said power source travels from one said active electrode through the tissue to another said active electrode and then returns to said ground electrode, and another amount of current from said power source travels directly from one said active electrode through the tissue to said ground electrode.

58. The system of claim 47, further comprising a circuit controlling operation of said power source.

59. The system of claim 49, further comprising:
a control circuit operably coupled to said impedance measuring circuit and controlling operation of said power source; and
wherein said control circuit discontinues the flow of power to said active electrodes when impedance measured by said impedance measuring circuit exceeds a threshold value.

60. The system of claim 59, wherein said impedance measuring circuit measures an initial impedance of the tissue, said control circuit sets the threshold value to equal said initial impedance, and said control circuit discontinues the flow of power to said active electrodes when impedance measured by said impedance measuring circuit exceeds said threshold value.

61. The system of claim 60, wherein said control circuit iterates through at least two power cycles where the control circuit sets the threshold value to equal an impedance value measured at the beginning of each said power cycle, applies power to said active electrodes, and discontinues power for a predetermined rest period when an impedance value measured by said impedance measuring circuit exceeds the threshold value.

62. The system of claim 61, wherein the control circuit discontinues power and terminates iteration through any further power cycles once power has been applied for a predefined duration regardless of an impedance value measured by said impedance measuring circuit.

63. The system of claim 47, further comprising at least one series electrode connected in series with one of said active electrodes.

64. The system of claim 1, wherein the total power applied to the tissue is less than 100 Watts.

65. The system of claim 1, wherein the total power applied to the tissue is less than 50 Watts.

66. A tissue coagulation system comprising:
a power source;
a plurality of active electrodes connected in parallel to said power source;
a RC circuit controlling a phase of voltage supplied by said power source connected to at least one said active electrode such that a different phase voltage is supplied to at least two different ones of said active electrodes; and
a ground electrode,
wherein said power source is electrically coupled to said ground electrode through the tissue.

67. The system of claim 66, wherein said active electrodes are mounted to a resilient housing.

68. The system of claim 66, further comprising an impedance measuring circuit operably connected to the power source, said impedance measuring circuit measuring the impedance of the tissue.

69. The system of claim 66, further comprising at least one thermocouple mounted to at least one of said active electrodes.

70. The system of claim 67, further comprising at least one thermocouple mounted to said housing.

71. The system of claim 66, wherein said plurality of active electrodes comprises two active electrodes and an area of one said active electrodes is larger than an area of the other said active electrode.

72. The system of claim 71, wherein the area of one said active electrode is three times the surface area of the other active electrode.

73. The system of claim 66, wherein:
said plurality of active electrodes comprise first and second active electrodes; and
said second active electrode comprises two segments which are adjacent to said first active electrode.

74. The system of claim 66, wherein said ground electrode is generally remote from said active electrodes.

75. The system of claim 66, wherein adjacent ones of said plurality of active electrodes are electrically insulated from one another.

76. The system of claim 66, further comprising:
a catheter sized to fit within the vascular system of a mammal, said catheter having an elongate tubular housing;
wherein said plurality of active electrodes are housed within said elongate tubular housing in an undeployed state.

77. The system of claim 66, wherein an amount of current from said power source travels from one said active electrode through the tissue to another said active electrode and then through the tissue to said ground electrode, and another amount of current from said power source travels directly from one said active electrode through the tissue to said ground electrode.

78. The system of claim 66, wherein an amount of current from said power source travels from one said active electrode through the tissue to another said active electrode and then returns to said ground electrode, and another amount of current from said power source travels directly from one said active electrode through the tissue to said ground electrode.

79. The system of claim 66, further comprising a circuit controlling operation of said power source.

80. The system of claim 68, further comprising:
a control circuit operably coupled to said impedance measuring circuit and controlling operation of said power source; and
wherein said control circuit discontinues the flow of power to said active electrodes when an impedance measured by said impedance measuring circuit exceeds a threshold value.

81. The system of claim 80, further comprising:
wherein said impedance measuring circuit measures an initial impedance of the tissue, said control circuit sets the threshold value to equal said initial impedance, and said control circuit discontinues the flow of power to said plurality of active electrodes when an impedance measured by said impedance measuring circuit exceeds said initial impedance.

82. The system of claim 81, further comprising:
wherein said control circuit iterates through at least two power cycles where the control circuit stores an impedance value measured at the beginning of each said power cycle as the threshold value, applies power to said at least two active electrodes, and discontinues power for a predetermined rest period when an impedance value measured by said impedance measuring circuit exceeds the threshold value.

83. The system of claim 82, wherein the control circuit discontinues power and terminates iteration through any further power cycles once power has been applied for a predefined duration regardless of an impedance value measured by said impedance measuring circuit.

84. The system of claim 66, wherein said RC circuit includes a plurality of RC circuits with one said RC circuit connected to each of said plurality of active electrodes such that the phase of voltage supplied to each said active electrode is different.

85. The system of claim 66, wherein said RC circuit includes a plurality of RC circuits with a different said RC circuit connected to adjacent ones of said active electrodes such that the phase of voltage supplied to adjacent active electrodes is unique.

86. The system of claim 66, wherein said power source comprises a plurality of power sources and adjacent ones of said plurality of active electrodes are connected to different said power sources.

87. The system of claim 66, further comprising:
a control circuit controlling operation of said power source and controlling operation of said RC circuit;
wherein said control circuit selectively controls said RC circuit so as to vary the amount of current from traveling from one said active electrode to another said active electrode.

88. The system of claim 86, wherein said control circuit selectively controls said RC circuit so as to vary over time the amount of current from traveling from one said active electrode to another said active electrode.

89. The system of claim 68, further comprising:
a circuit controlling operation of said power source and controlling operation of said RC circuit;
wherein said control circuit selectively controls said at RC circuit so as to vary, in response to a detected impedance, the amount of current from traveling from one said active electrode to another said active electrode.

90. The system of claim 68, further comprising:
a circuit controlling operation of said power source and controlling operation of said RC circuit;
wherein said control circuit selectively controls said RC circuit so as to vary, in response to a detected temperature, the amount of current from traveling from one said active electrode to another said active electrode.

91. A tissue coagulation welding system comprising:
a plurality of active electrodes; and
a plurality of power sources, with one said power source electrically coupled to each said active electrode, a frequency of voltage supplied by at least two of said plurality of power sources being different, such that the voltage drop across one said active electrode is different from the voltage drop across a different said active electrode;
wherein each said power source is electrically coupled to a ground electrode through the tissue.

92. The system of claim 91, wherein said active electrodes are mounted to a resilient housing.

93. The system of claim 91, further comprising an impedance measuring circuit operably connected to the power source, said impedance measuring circuit measuring the impedance of the tissue.

94. The system of claim 91, further comprising at least one thermocouple mounted to at least one of said active electrodes.

95. The system of claim 92, further comprising at least one thermocouple mounted to said housing.

96. The system of claim 91, wherein said plurality of active electrodes comprise two active electrodes and an area of one said active electrodes is larger than an area of the other said active electrode.

97. The system of claim 96, wherein the area of one said active electrode is three times the surface area of the other active electrode.

98. The system of claim 91, wherein:
said plurality of active electrodes comprise first and second active electrodes; and
said second active electrode comprises two segments which are adjacent to said first active electrode.

99. The system of claim 91, wherein said ground electrode is generally remote from said active electrodes.

100. The system of claim 91, wherein adjacent ones of said plurality of active electrodes are electrically insulated from one another.

101. The system of claim 91, further comprising:
a catheter sized to fit within the vascular system of a mammal, said catheter having an elongate tubular housing;
wherein said plurality of active electrodes are housed within said elongate tubular housing in an undeployed state.

102. The system of claim 90, wherein an amount of current from said power source travels from one said active electrode through the tissue to another said active electrode and then through the tissue to said ground electrode, and another amount of current from said power source travels directly from one said active electrode through the tissue to said ground electrode.

103. The system of claim 90, wherein an amount of current from said power source travels from one said active electrode through the tissue to another said active electrode and then returns to said ground electrode, and another amount of current from said power source travels directly from one said active electrode through the tissue to said ground electrode.

104. The system of claim 91, further comprising a circuit controlling operation of said power source.

105. The system of claim 93, further comprising:
a control circuit operably coupled to said impedance measuring circuit and controlling operation of said power source; and
wherein said control circuit discontinues the flow of power to said active electrodes when an impedance measured by said impedance measuring circuit exceeds a threshold value.

106. The system of claim 104, further comprising:
wherein said impedance measuring circuit measures an initial impedance of the tissue, said control circuit sets the threshold value to equal said initial impedance, and said control circuit discontinues the flow of power to said plurality of active electrodes when an impedance measured by said impedance measuring circuit exceeds said initial impedance.

107. The system of claim 106, further comprising:
wherein said control circuit iterates through at least two power cycles where the control circuit stores an impedance value measured at the beginning of each said power cycle as the threshold value, applies power to said at least two active electrodes, and discontinues power for a predetermined rest period when an impedance value measured by said impedance measuring circuit exceeds the threshold value.

108. The system of claim 107, wherein the control circuit discontinues power and terminates iteration through any further power cycles once power has been applied for a predefined duration regardless of an impedance value measured by said impedance measuring circuit.

109. A tissue coagulation system comprising:
a power source;
a plurality of active electrodes connected in parallel to said power source;
at least one diode connected in series with one of said plurality of active electrodes such that the voltage drop across one of said active electrodes is different from the voltage drop across another of said active electrodes;
a ground electrode;
wherein said power source is electrically coupled to said ground electrode through the tissue.

110. The system of claim 109, wherein said active electrodes are mounted to a resilient housing.

111. The system of claim 109, further comprising an impedance measuring circuit operably connected to said power source, said impedance measuring circuit measuring the impedance of the tissue.

112. The system of claim 109, further comprising at least one thermocouple mounted on at least one of said active electrodes.

113. The system of claim 110, further comprising at least one thermocouple mounted to said housing.

114. The system of claim 109, wherein a surface area of one of said active electrodes is larger than a surface area of another of said electrodes.

115. The system of claim 114, wherein said plurality of active electrodes comprise two active electrodes with one active electrode having a surface area at least three times as large as the surface area of the other active electrode.

116. The system of claim 115, wherein said plurality of active electrodes comprise two active electrodes with one of said two active electrodes comprising two segments which are adjacent to the other said two active electrodes.

117. The system of claim 109, wherein said ground electrode is generally remote from said active electrodes.

118. The system of claim 109, wherein adjacent ones of said electrodes are generally electrically insulated from one another such that current traveling between electrodes generally passes through tissue.

119. The system of claim 109, further comprising:
a catheter sized to fit within the vascular system of a mammal, said catheter having an elongate tubular housing;
wherein said active electrodes are housed within said elongate tubular housing in an undeployed state.

120. The system of claim 109, wherein an amount of current from said power source travels from one said active electrode through the tissue to another said active electrode and then through the tissue to said ground electrode, and another amount of current from said power source travels directly from one said active electrode through the tissue to said ground electrode.

121. The system of claim 109, further comprising a circuit controlling operation of said power source.

122. The system of claim 111, further comprising:
a control circuit operably coupled to said impedance measuring circuit and controlling operation of said power source;
wherein said control circuit discontinues the flow of power to said active electrodes when impedance measured by said impedance measuring circuit exceeds a threshold value.

123. The system of claim 122, wherein said impedance sets the threshold value to equal an initially measured value, initiates a flow of power to said active electrodes, and discontinues the flow of power to said active electrodes when impedance measured by said impedance measuring circuit exceeds said threshold value.

124. The system of claim 123, wherein said control circuit iterates through at least two power cycles where the control circuit sets the threshold value as an impedance value measured at the beginning of each said power cycle, initiates a flow of power to said active electrodes, and discontinues power for a predetermined rest period when an impedance value measured by said impedance measuring circuit exceeds the threshold impedance value stored at the beginning of that power cycle.

125. The system of claim 124, wherein the control circuit discontinues power and terminates iteration through any further power cycles once power has been applied for a predefined duration regardless of an impedance value measured by said impedance measuring circuit.

126. The system of claim 109, further comprising a diode control circuit controlling said at least one diode to control the path of the current flow between said at least two active electrodes.

127. The system of claim 122, wherein:
said plurality of active electrodes comprises N-number of active electrodes;
said at least one diode comprises N-number of diodes, with one said diode connected in series with each of said N-number of active electrodes; and
said control circuit controls said diodes so as to control the relative flow of current between said active electrodes.

128. The system of claim 127, wherein said control circuit includes a diode control circuit controlling said plurality of diodes to control the path of the current flow between said active electrodes.

129. The system of claim 128, wherein said control circuit discontinues the flow of power to said active electrodes when impedance measured by said impedance measuring circuit exceeds a threshold value.

130. The system of claim 129, wherein said impedance measuring circuit measures an initial impedance of the tissue, and said control circuit discontinues the flow of power to said active electrodes when impedance measured by said impedance measuring circuit exceeds said initial impedance.

131. The system of claim 130, wherein said control circuit iterates through at least two power cycles, said control circuit stores an impedance value measured at the beginning of each said power cycle, applies power to said active electrodes, and discontinues power to said active electrodes for a predetermined rest period when an impedance value measured by said impedance measuring circuit exceeds the impedance value stored at the beginning of that power cycle.

132. The system of claim 131, wherein the control circuit discontinues power and terminates iteration through any further power cycles one power has been applied for a predefined duration regardless of an impedance value measured by said impedance measuring circuit.

133. An apparatus for coagulating tissue comprising:
an elongate flexible member having a proximal end and a distal end;
a plurality of active electrodes disposed near the distal end of the elongate flexible member, said plurality of electrodes adapted to be coupled in parallel to a power source, said plurality of electrodes also adapted so that a resistor connected in series with one of said plurality of electrodes members results in a voltage drop across said one electrode different from a second voltage drop across another of said plurality of electrodes.

134. The apparatus of claim 133, further comprising a resilient housing near the distal end of said elongate flexible member and wherein said plurality of electrodes are coupled with said resilient housing.

135. The apparatus of claim 133, further comprising a thermocouple coupled to one of said plurality of electrodes.

136. The apparatus of claim 134, further comprising a thermocouple coupled with said resilient housing.

137. The apparatus of claim 133, wherein a surface area of one of said plurality of electrodes is larger than a surface area of another of said electrodes.

138. The apparatus of claim 133, wherein said plurality of electrodes comprise two active electrodes with one active electrode having a surface area at least three times as large as the surface area of the other active electrode.

139. The apparatus of claim 133, wherein said plurality of electrodes comprise two active electrodes with one of said two active electrodes comprising two segments which are adjacent to the other said two active electrodes.

140. The apparatus of claim 133, wherein adjacent ones of said electrodes are generally electrically insulated from one another such that current traveling therebetween travels through tissue.

141. An apparatus for coagulating tissue comprising:
an elongate flexible member having a proximal end and a distal end;
a plurality of active electrodes disposed near the distal end of the elongate flexible member and coupleable in parallel to a power source, said plurality of electrodes also adapted to be coupled to a RC circuit controlling a phase of voltage supplied to at least one of said plurality of electrodes such that a different phase voltage can be supplied to at least two different ones of said plurality of electrodes.

142. The apparatus of claim 141, further comprising a resilient housing near the distal end of said elongate flexible member and wherein said plurality of electrodes are coupled with said resilient housing.

143. The apparatus of claim 141, further comprising a thermocouple coupled to one of said plurality of electrodes.

144. The apparatus of claim 142, further comprising a thermocouple coupled with said resilient housing.

145. The apparatus of claim 141, wherein a surface area of one of said plurality of electrodes is larger than a surface area of another of said plurality of electrodes.

146. The apparatus of claim 141, wherein said plurality of electrodes comprise two active electrodes with one active electrode having a surface area at least three times as large as the surface area of the other active electrode.

147. The apparatus of claim 141, wherein said plurality of electrodes comprise two active electrodes with one of said two active electrodes comprising two segments which are adjacent to the other of said two active electrodes.

148. The apparatus of claim 141, wherein adjacent ones of said electrodes are generally electrically insulated from one another such that current traveling therebetween travels through tissue.

149. An apparatus for coagulating tissue comprising:
an elongate flexible member having a proximal end and a distal end; and
a plurality of active electrodes disposed near the distal end of said elongate flexible member, said plurality of electrodes adapted to be coupled with two or more power sources such that a frequency of voltage supplied by said two or more power sources are different and that the voltage drop across one of said plurality of electrodes is different from the voltage drop across a different of said plurality of electrodes.

150. The apparatus of claim 149, further comprising a resilient housing near the distal end of said elongate flexible member and wherein said plurality of electrodes are coupled with said resilient housing.

151. The apparatus of claim 149, further comprising a thermocouple coupled to one of said electrodes.

152. The apparatus of claim 150, further comprising a thermocouple coupled with said resilient housing.

153. The apparatus of claim 149, wherein a surface area of one of said plurality of electrodes is larger than a surface area of another of said plurality of electrodes.

154. The apparatus of claim 149, wherein said plurality of electrodes comprise two active electrodes with one active electrode having a surface area at least three times as large as the surface area of the other active electrode.

155. The apparatus of claim 149, wherein said plurality of electrodes comprise two active electrodes with one of said two active electrodes comprising two segments which are adjacent to the other of said two active electrodes.

156. The apparatus of claim 149, wherein adjacent ones of said electrodes are generally electrically insulated from one another such that current traveling therebetween travels through tissue.

157. An apparatus for coagulating tissue comprising:
an elongate flexible member having a proximal end and a distal end; and
a plurality of active electrodes disposed near the distal end of the elongate flexible member, said plurality of electrodes adapted to be coupled in parallel to a power source, said plurality of electrodes also adapted so that a diode connected in series with one of said plurality of electrodes members results in a voltage drop across said one electrode different from a second voltage drop across another of said plurality of electrodes.

158. The apparatus of claim 157, further comprising a resilient housing near the distal end of said elongate flexible member and wherein said plurality of electrodes are coupled with said resilient housing.

159. The apparatus of claim 157, further comprising a thermocouple coupled to one of said plurality of electrodes.

160. The apparatus of claim 157, further comprising a thermocouple coupled with said resilient housing.

161. The apparatus of claim 157, wherein a surface area of one of said plurality of electrodes is larger than a surface area of another of said electrodes.

162. The apparatus of claim 157, wherein said plurality of electrodes comprise two active electrodes with one active electrode having a surface area at least three times as large as the surface area of the other active electrode.

163. The apparatus of claim 157, wherein said plurality of electrodes comprise two active electrodes with one of said two active electrodes comprising two segments which are adjacent to the other said two active electrodes.

164. The apparatus of claim 157, wherein adjacent ones of said electrodes are generally electrically insulated from one another such that current traveling therebetween travels through tissue.

165. A method for coagulating tissue, the method comprising:
bringing a treatment apparatus to a tissue treatment site, the treatment apparatus having a proximal end, a distal end and a first and a second active electrode near the distal end;
positioning the first and the second electrodes into apposition with tissues of the tissue treatment site so that the treatment apparatus may effectively coagulate the tissue; and
applying a first potential to the first electrode and a second potential lower than the first potential to the second electrode so that current flows from the first energy transmission member through the tissue to the second energy transmission member and then through the tissue to a ground electrode, and current also flows from the first electrode through the tissue to the ground electrode.

166. The method of claim 165, wherein current also flows from the first electrode through the tissue to the second electrode and returns to the ground electrode.

167. The method of claim 165, further comprising measuring impedance of the tissue.

168. The method of claim 167, wherein the potential applied to the first and second electrodes is controlled based on the measured tissue impedance.

169. The method of claim 165, further comprising measuring temperature of the tissue with a thermocouple disposed on either the first or second electrodes.

170. The method of claim 169, wherein the potential applied to the first and second electrodes is controlled based on the measured tissue temperature.

171. The method of claim 165, further comprising deploying the first and second electrodes from a catheter.

172. The method of claim 165, wherein applying the second potential comprises providing a resistor in series with the second electrode so that the second potential is lower than the first potential.

173. The method of claim 165, wherein applying the first potential and the second potential comprises providing two power supplies.

174. The method of claim 165, wherein applying the second potential comprises providing a RC circuit in series with the second electrode so that the second potential is out of phase with the first potential.

175. The method of claim 165, wherein applying the second potential comprises providing the second potential at a frequency different than the frequency of the first potential.

176. The method of claim 165, wherein applying the second potential comprises providing a diode in series with the second electrode so that the second potential is lower than the first potential.
